(19) United States
(12) Patent Application Publication
(10) Pub. No.: US 2025/0221388 A1
(43) Pub. Date: Jul. 10, 2025

(54) SYSTEMS AND PROCESSES FOR AQUACULTURE CROP ROTATION

(71) Applicant: NESTE OYJ, Espoo (FI)

(72) Inventors: Jeffrey KANEL, Porvoo (FI); Cecil CHURN, III, Porvoo (FI); Charles HARRIS, Porvoo (FI); David BRYANT, Porvoo (FI)

(21) Appl. No.: 18/724,518

(22) PCT Filed: Dec. 30, 2022
(86) PCT No.: PCT/FI2022/050878

§ 371 Date: Jun. 26, 2024

Related U.S. Application Data
(60) Provisional application No. 63/295,543, filed on Dec. 31, 2021.

(30) Foreign Application Priority Data
Mar. 30, 2022 (FI) 20225275

(51) Int. Cl. A01K 63/10; A01G 33/00; A01K 61/10; A01K 61/51; A01K 61/54; A01K 61/59; A01K 63/00

(52) U.S. Cl. CPC A01K 63/10; A01G 33/00; A01K 63/003; A01K 61/10; A01K 61/51; A01K 61/54; A01K 61/59

(57) ABSTRACT

A process, system, and use of the system are disclosed, which enable the successive growth of aquatic animals and algae within the same aquaculture pond and optionally reduction of infestation species in ponds bottom solids of the aquaculture pond.

### FIELD

The present application relates to processes and systems for the growth of aquatic animals and algae, and in particular to processes and systems for the growth of aquatic animals and algae within the same aquaculture pond.

### BACKGROUND AND OBJECTS

Crop rotation methods have been applied in agriculture since Roman times (Walker 2010), but there is scant literature on the rotation of aquacultural crops. Aquaculture is the farming of aquatic organisms such as algae, microalgae, shellfish, shrimp, and fish. In aquaculture, marine and freshwater populations are cultivated under controlled conditions.

Crop rotation is one of the oldest and most effective agricultural control strategies. It means the planned order of specific crops planted on the same field. It also means that the succeeding crop belongs to a different family than the previous one. The planned rotation may vary from 2 or 3 years or longer periods. Crop rotation avoids a decrease in soil fertility, as growing the same crop in the same place for sequential years may disproportionately deplete the soil of certain nutrients.

Crop rotation is further used to control predators, competitors, pests, and diseases that can become established in the soil over time. The changing of crops in a sequence tends to decrease the population level of pests. Some insect pests and disease-causing organisms are host specific. For example, rice stem borer feeds mostly on rice. If rice is not rotated with other crops belonging to a different family, the problem continues, as food is always available to the pest. However, if legumes are planted as the next crop, then corn, then beans, then bulbs, the insect pest will likely die due to absence of food.

Aquaculture is the farming of aquatic organisms such as algae, microalgae, shellfish, shrimp, and fish. In aquaculture, marine and freshwater populations are cultivated under controlled conditions. Aquacultural farming, however, suffers from problems similar to agricultural farming. For example, aquatic animal wastes and unutilized feed residues accumulate as organic and mineralized debris in production pond bottoms. This provides a substrate for microbial pathogens with the potential to decimate growth and survival of the targeted species, thereby negatively impacting economic productivity of the enterprise.

Draining the aqueous growth media from the pond is not entirely effective in eradicating bacterial pathogens, since many microbial pathogens can survive for extended periods of time in pond bottoms soils to negatively impact survival rates during the next production cycle.

There is also increasing interest in aquacultural algal farming of algae for a plethora of sustainable activities, such as a source of renewable energy, as a source of protein, as a mode to safely and efficiently capture carbon dioxide from the atmosphere for carbon sequestration, and as a renewable source of chemical intermediates (Wiffels 2010).

Unfortunately, as the aquaculture industry continues to grow, and yields are increased, the problems associated with aquaculture waste management increase proportionally. It is not only the sheer volume of waste material from the processing of farmed aquatic species, but also the potential for the spread of bacterial and viral pathogens through untreated wastewater discharge into natural environments that presents a challenge to the industry. Disease mitigation is a management problem exacerbated by the relatively high stocking densities and the close proximity of aquaculture wastewater discharged into water resources.

In an effort to maximize productivity of an aquaculture pond, such as one for growing shrimp, stocking and feeding rates are often increased. Excessive buildup of waste with metabolic byproducts produced by feeding increases the biological oxygen demand for the system, resulting in less dissolved oxygen available for the target species. Deterioration of aerobic conditions in the system creates stress factors that reduce growth and survival rates, and the potential for manifestations of disease and subsequent mortality. Wastewater effluent from the process of water exchange and harvesting contains excessive nutrients, suspended solids, organic matter, and potential pathogenic organisms. The effluent water released to coastal waters joins the water supply for neighboring aquaculture ponds (Boyd 1992) and can transfer bacterial and/or viral pathogens.

Both shellfish and fish are cultivated in intensive production systems where stress may be more prevalent and the manifestation of disease more critical. The following paragraphs cite examples for shrimp aquaculture, but similar examples may be found for shellfish and fish aquaculture systems.

With higher shrimp stocking densities, the incident of disease may be amplified, in some cases severely enough to decimate a pond's shrimp population in just a few days. In some countries, a significant portion of the shrimp industry has been impacted. Accute hepatopancreatic necrosis “EMS” (early mortality disease) was first detected in Mexico during the 2013 shrimp production cycle and losses amounted to a decrease in industry production from 100,000 tonnes in year 2012 to less than 60,000 tonnes (Fegan, Second FAO Symposium on AHPND Bangkok, 2016).

In Mexico's Gulf of California aquaculture industry, organic debris accumulates in shrimp production ponds to such an extent that productivity may be negatively impacted after 3-4 years when no remediation management strategy is implemented. Currently, the only way to treat these ponds is to: 1) drain the pond, 2) allow the soil to dry, 3) disc the bottom of the pond to about 30 cms deep for exposing organic debris to solar UV and atmospheric air, 4) allow time for the accumulated debris to degrade and become oxidized, and 5) refill the ponds for restocking. In the region, this process is performed during the “dry season” between November and April, as shrimp are harvested in November and restocked in March-April for an 8-month production cycle. This protocol occurs in the arid climate of the coastal Sonora desert but in many regions of the world, a “dry season” and the time between harvest and restocking do not correspond. For example, in South East Asia, calcium hydroxide is added to ponds for soil pH stabilization in managing organic debris.

In 1988, Taiwan, then the top producer of industrial shrimp, lost 75 percent of its harvest to a virus called Monodon baculovirus. China then became the top producer, until it was hit with disease caused by hypodermal and hematopoietic virus (Lightner 2003). In 1999, Ecuador lost half of its crop to Taura syndrome and white spot syndrome virus. The shrimp industries of Indonesia, India, Honduras and Mexico also faced significant disease outbreaks in the 1990s.

Control of diseases is a primary impediment to growth of the shrimp aquaculture industry. Historically, shrimp diseases of most importance have been: Taura syndrome virus (TSV), white spot syndrome virus (WSSV), and the necrotizing hepatopancreatitis bacterium (NHP-B).

Taura Syndrome Virus (TSV) has had a devastating economic impact on the shrimp aquaculture industry. TSV is a 30 to 32 nm, icosahedral virus particle containing positive-sense, single-stranded RNA of about 10.2 kb in length. TSV is a member of the “picornavirus superfamily,” the Family Dicistroviridae and the Genus Cripavirus.

White spot syndrome virus (WSSV) is the most virulent viral pathogen of penaeid shrimp. WSSV is an ovoidal, enveloped particle about 80-120 nm wide by 250-380 nm long with a small tail-like appendage at one end. The virion's genome consists of a single, circular, double-stranded DNA molecule of about 300 kb in length. The virus belongs to the viral Family Nimaviridae and the Genus Whispovirus. There have been several publications on how to deal with WSSV, and Desal (U.S. Pat. No. 6,440,466) disclosed a composition of plant derived extracts to treat it.

White spot syndrome virus is a leading disease affecting shrimp yields. White spots appear on shrimp exoskeleton and their bodies to steadily decompose in as few as 10 days. White spot is often accompanied by vibriosis, which is caused by Vibrio bacteria. These bacteria exist naturally in coastal waters and infect shrimp when they become stressed by problems like poor water quality, another disease or crowding. Vibrio bacteria are especially problematic: if humans eat the infected shrimp, they can become sick with gastroenteritis (caused by Vibrio parahaemolyticus), cholera (caused by Vibrio cholerae) or suffer from fatal septic shock (caused by Vibrio vulnificus) (Blake, et al., 1979).

In addition to White Spot, a variety of other viral transmitted diseases can decimate shrimp farms as summarized in Table 1 after Walker (2010), Viral Diseases of Shrimp and Table 2, Shrimp Species and Disease Symptoms (Gill 2000).

Other viral pathogens have been identified in shrimp farms around the world and have caused devastation to the industry because of their highly contagious nature. There are no known cures for viral pathogens making prevention the only means of reducing the economic impact. (Walker 2010).

Viruses are DNA or RNA encased in a protein capsid. Viruses can be classified as naked or enveloped. The naked viruses have their DNA or RNA surrounded by a simple protein coating. Enveloped viruses are surrounded by phospholipids that they steal from the cells that they parasitize. Enveloped viruses can be rendered harmless when their viral envelope is destroyed, because the virus no longer has the recognition sites necessary to identify and attach to host cells. Enveloped viruses have protein probes projecting through their phospholipid coating. There have been several approaches to deal with viral agents, and Sagawa (U.S. Pat. No. 6,518,317) disclosed some specific organic antiviral agents.

In addition to viral diseases, shrimp colonies are susceptible to a variety of bacterial diseases. These are summarized in Table 3, Bacterial Diseases of Shrimp.

Bacteria are natural microflora of seawater and are present in shrimp pond water. The accumulation of unutilized feed and shrimp fecal matter supports the multiplication of bacteria. Bacterial infections of shrimp are primarily stress related. Adverse environmental conditions, sudden osmotic changes or mechanical injuries are important factors in shrimp bacterial infections. Intensive shrimp farming imposes stress on shrimp and makes them more susceptible to disease.

Bacterial diseases include Vibriosis, Necrotizing Hepatopancreatitis, Zoea II Syndrome, Mycobacteriosis and Rickettsial Disease. The shrimp farming industry has been dealing and managing Vibriosis for decades. Acute Hepatopancreatin Necrosis Disease (AHPND), causative agent of Vibrio parahaemolyticus with a common name of “Early Mortality Syndrome” (EMS) almost destroyed the industry. It is the number one bacterial pathogen impacting today's shrimp culture industry worldwide.

Vibriosis is also known as Blackshell Disease, Septic Hepatopancreatic Necrosis, Tail Rot, Brown Gill Disease, Swollen Hindgut Syndrome, Firefly Disease and Luminous Bacterial Disease.

Necrotizing Hepatopancreatitis, NHP, also known as Texas Necrotizing Hepatopancreatitis (TNHP), Granulamatous hepatopancreatitis, Texas Pond Mortality Syndrome (TPMS), Peru Necrotizing Hepatopancreatitis (PNHP) is a severe bacterial disease affecting penaeid shrimp aquaculture. NHP results in significant mortalities and devastating losses to shrimp crops. Elevated salinity and temperature above that in typical shrimp aquaculture appear to be stress factors for the shrimp and are associated with NHP outbreaks. The magnitude of the elevated salinities in shrimp aquaculture are just slightly elevated over seawater, with values of about 4 to 5 wt-%

Zoea II Syndrome has no known treatment.

Mycobacteriosis, also known as Mycobacterium Infection of Shrimp and Shrimp Tuberculosis has no proven treatment but prolonged use of a combination of antimicrobials is thought to be effective.

Rickettsial Disease has no proven treatment.

Management protocols are being developed for Vibriosis and AHPND. However, there are always new diseases impacting the international shrimp farming industry. One example is the obligate microsporidian parasite Enterocytozoon hepatopenaei (EHP) is the causative agent of hepatopancratic microsporidosis and reported in association with “white feces”-syndrome that causes slow growth, morbidity and or mortality which has had a severe economic impact on Asian shrimp production [Loc Tran. Second FAO Symposium on AHPND Bangkok 2016].

Natural infections have been recorded from black tiger shrimp (Penaeus monodon), Kuruma shrimp (P. japonicus), Chinese white shrimp (P. chinensis), banana prawns (P. merguiensis and P. inducus), white shrimp (P. vannamei) and other penaeids.

It is reasonable to assume that all cultured penaeids would be susceptible to infection. There are about 100 penaeid shrimp species, of which a dozen of Penaeus spp. and Metapenaeus spp. have commercial value. The shrimp types cultured are: Penaeus chinensis, P. monodon, P. japonicus, P. merguinsis, P. penicillatus, Metapenaeus ensis and P. vannamei. Natural infections also occur in many species of decapods (crabs, crayfish, lobsters and shrimp) or other crustaceans, although often not lethal.

Traditional commercial shrimp operations discharge untreated polluted water and waste products directly onto surrounding lands and into adjacent waterways. The nutrients from shrimp operations discharge can lead to a decline in local water quality. They contain plant nutrients, such as nitrogen and phosphorus that accelerate phytoplankton growth and eutrophication of the water into which it is discharged. There has been much effort devoted to alleviation of eutrophication; the only solution found so far is to reduce the pollution load. A high concentration of extensive (traditional) or intensive shrimp farms contributes to degradation of coastal waters.

Excess nutrients including nitrogen and phosphorus and organic matter discharged from the shrimp production systems through pond management practices of routine water exchange leads to eutrophication and worsening of the environment. Changing the pond water in order to maintain the water quality in the ponds will result in the discharge of wasted feed, fecal material and nutrients to the environment, worsening marine water quality. The polluted seawater may become the sources of the pollution and diseases in other shrimp ponds. In addition, drugs such as antibiotic and disinfectors used to control shrimp diseases, may accumulate in the shrimp, thus affecting the seafood quality.

The conversion ratio of feed input into a traditional pond aquaculture system and shrimp biomass harvested from the system, has an average range of 1.6-2.0:1. Approximately 35% of the total feed applied to a system is excreted into the water column to accumulate as organic and mineralized residues. As much as 75% of the nitrogen and phosphorous component of shrimp feeds is discharged into natural environments in wastewater effluents from production facilities. Routine hydraulic exchange in traditional extensive shrimp pond aquaculture averages 5-10% of pond volume per day. Daily water exchange is required to mediate the negative impact of water quality degradation that results from accumulation of metabolic wastes during normal growth cycle feeding activities.

Concentrated traditional shrimp farming has caused a large amount of coastal pollution in many parts of the world. For example, many shrimp farms in central and eastern Thailand have failed due to pollution, according to Vaiphasa (2007). In other areas where coastal pollution has become critical, historically excessive amounts of chemical additives and antibiotics have been used to keep diseases from causing excessive shrimp mortality. In many cases, these practices have not had success in controlling disease.

Therefore, international aquaculture industry traditionally has not practiced effective management protocols to control the manifestation of microbial related diseases for commercial shrimp, shellfish, and fish production, without the use of antibiotics or reducing stocking densities, or applying significant water treatment techniques, other methods, or combinations known in the art.

Management control of disease manifestation in pond systems also has the benefit of mitigating the impact of organic loading and pathogens in the farm wastewater effluent contaminating the intake water of adjacent or regional aquaculture production facilities, a common problem in tropical coastal zones that become saturated with shrimp farm development.

TABLE 1
Exemplary Viral Diseases of Shrimp

| | | | |
|---|---|---|---|
| | | Taxonomic | Naked or |
| Abbreviation | Genome | classification¹ | Enveloped? |

DNA viruses

| | | | | |
|---|---|---|---|---|
| Monodon baculovirus | MBV | dsDNA | Baculoviridae | Enveloped |
| Baculoviral midgut | BMNV | dsDNA | Baculoviridae | Enveloped |
| gland necrosis virus | | | | |
| White spot syndrome | WSSV | dsDNA | Nimaviridae, | Enveloped |
| virus | | | Whispovirus | |
| Infectious hypodermal | IHHNV | ssDNA | Parvoviridae, | Naked |
| and haematopoietic | | | Densovirus | |
| necrosis virus | | | | |
| Hepatopancreatic | HPV | ssDNA | Parvoviridae, | Naked |
| parvovirus | | | Densovirus | |

RNA viruses

| | | | | |
|---|---|---|---|---|
| Yellow head virus | YHV | (+) | Nidovirales, | Enveloped |
| | | ssRNA | Roniviridae, Okavirus | |
| Taura syndrome virus | TSV | (+) | Picornavirales, | Naked |
| | | ssRNA | Dicistroviridae | |
| Infectious | IMNV | dsRNA | Totivirus | Naked |
| myonecrosis virus | | | (unclassified) | |
| Macrobrachium | MrNV | (+) | Nodavirus | Naked |
| rosenbergii nodavirus | | ssRNA | (unclassified) | |
| (White Tail Disease) | | | | |
| Laem-Singh virus | LSNV | (+) | Luteovirus-like | Naked |
| | | ssRNA | (unclassified) | |
| Mourilyan virus | MoV | (−) | Bunyavirus-like | Enveloped |
| | | ssRNA | (unclassified) | |

TABLE 2
Exemplary Shrimp Species and Disease Symptoms (Gill 2000)

| Virus | Species Affected | Disease/Symptom |
|---|---|---|
| Taura Syndrome | Penaeus | Lesions in cuticle, gills, |
| Virus (TVS) | vannamei | digestive tract and |
| | | appendages. |
| White Spot | P. vannamei, P. | White spots on exoskeleton, |
| Baculovirus | monodon, P. | appendages and other body |
| (WSBV) | japonicus, P. | parts. Lethargy and red |
| | indicies, P. | discolouration of |
| | chenensis | hepatopancreas. |
| Yellowhead | P. vannamei, P. | Yellow discolouration of |
| Virus (YHV) | monodon | carapace, gills or |
| | | hepatopancreas. Hypertrophy |
| | | of nucleii in cells of lymphoid |
| | | organ, hepatopancreas, gills |
| | | and heart. |
| Infectious | P. stryloristris, P. | Stunted growth, deformities, |
| Hypodermal and | monodon | cellular inclusion bodies. |
| Haematopoietic | (unaffected | Affects cuticular epidermis, |
| Necrosis Virus | carrier) | haematocytes, haemopoietic |
| (IHHNV) | | organs and connective tissue. |

TABLE 3
Exemplary Bacterial Diseases of Shrimp

| Disease | a.k.a. | Bacterium | Treatment |
|---|---|---|---|
| Vibriosis | Known as Blackshell | Numerous | Medicated feed at |
| | Disease, Septic | etiological agents: | 4 g/kg oxytet, |
| | Hepatopancreatic | V. harveyi, V. | nitrofurizolidone, |
| | Necrosis, Tail Rot, | vulnificus, V. | sarafloxathin |
| | Brown Gill Disease, | parahaemolyticus, | (Sarafin); |
| | Swollen Hindgut | V. alginolyticus, | Formalin at 10- |
| | Syndrome, Firefly | V. penaeicida, V. | 25 ppm; |
| | Disease and Luminous | anguillarum, V. | Malachite Green |
| | Bacterial Disease | splendidus, V. | at 5-10 ppb; |
| | | damsela | EDTA at 10-50 |
| | | | ppm; Furanace at |
| | | | 1 ppm; |
| | | | Chloramphenical |
| | | | at 1-10 ppm; |
| | | | OTC at 1-10 |
| | | | ppm |
| Necrotizing | Texas Necrotizing | New genus of the | Use of oxytet at 4 |
| Hepatopancreatitis | Hepatopancreatitis | Protobacteria | g/kg (4,000 ppm), |
| | (TNHP), | (alpha) group | OTC at 0.5-2 kg |
| | Granulamatous | | per 1000 kg of |
| | hepatopancreatitis, | | feed |
| | Texas Pond Mortality | | |
| | Syndrome (TPMS), | | |
| | Peru Necrotizing | | |
| | Hepatopancreatitis | | |
| | (PNHP) | | |
| Zoea II Syndrome | | 1997: agent | No known |
| | | determined to be | treatment |
| | | intracellular | |
| | | bacteria (found by | |
| | | TEM in HP) | |
| Mycobacteriosis | Mycobacterium | Mycobacterium | No known |
| | Infection of Shrimp | marinum and M. | treatment but |
| | and Shrimp | fortuitum | prolonged use of a |
| | Tuberculosis | | combination of |
| | | | antimicrobials |
| | | | thought to be |
| | | | effective |
| Rickettsial Disease | Rickettsial infection of | Rickettsia or | None reported; |
| | Penaeid shrimp | Rickettsia-like | tetracycline |
| | | microorganisms | |

For the successful management of shrimp farms, the factors of predators, competitors, and pests also need to be addressed. These issues have been well addressed by Harry (1978), and Section 9 of that work is excerpted herein. It is desirable to have a sustainable method to control the predators, competitors, and pests.

For shrimp and shellfish, predators include fish, crabs, birds, insects, snakes, otters, and lizards. Several of these predators will be discussed in detail for shrimp aquaculture, and they can be applied as reasonable to other shellfish and fish aquaculture systems.

For shrimp, and shellfish competitors include snails, fish, crabs, birds and other species of shrimp.

For shrimp and shellfish, pests include crabs, burrowing shrimp such as Thalassina, organisms that degrade wood, mud worm egg cases, and shells.

Fish can act as a predator or as a competitor to shrimp and shellfish. The most efficient control method is prevention of allowing them to enter the shrimp ponds. First, proper maintenance of the ponds is necessary to prevent fish from entering through leaks in the dikes and weir boxes. Second, drying the pond bottom thoroughly before stocking with shrimp will eliminate fish. Third, screening water as it enters the pond is an important method to control fish entry into the shrimp ponds. The screens must be sufficiently fine to prevent transport of fish eggs and larvae as well as the adult fish. Typically, a plastic screen with a hole size of 0.2 to 0.5 millimeters is recommended, but this fine mesh is easily stopped up unless a series of screens are used. Optionally, a bag filter may be used to increase the filtration area. If a bag filter is used, then the discharge end of the bag may be connected to a floating screen box where trash will collect and can be removed by a dip net.

Crabs are one of the most destructive animals in a shrimp pond according to Harry (1978). The swimming crabs of the Family Portunide are especially destructive to shrimp—and need to be trapped. Burrowing crabs are the source of water leakage through pond dikes. The insecticide “Sevin” historically was used for killing crabs, but it was also toxic to shrimp.

Snails compete with the shrimp for food in a pond—and according to Harry (1978) most operators feel that production is lowered with increased numbers of snails. Several commercial preparations were historically used to kill snails that include “Brestan,” “Aquatin,” and “Bayluscide.” However, these compounds are no longer in use.

Wading birds are a predator to shrimp. If the water in the pond is kept deep enough and colored with a growth of phytoplankton, the most wading birds cannot see the bottom and will not land. This can be an effective means to control wading bird predation but does not control diving birds such as cormorants.

For other pests, such as insects, all but one of the pesticides used globally in shrimp production are banned for use in U.S. shrimp farms. Only a diluted form of formaldehyde, called formalin, is approved for U.S. shrimp farms. The FDA is capable of testing imported shrimp for residues of 360 pesticides, and shipments over the legal limit may be refused.

For organisms that degrade wood, this is a major problem for water control structures. This risk can be reduced by constructing water control structures of concrete. However, if wood construction is used, or if wood is used in concrete control structures as weir boards, then the following wood degrading organisms should be considered: Mollusca (Teredinidae or shipworms, Pholadidae or piddocks), Crustacea (Isopoda), and Fungi. The Mollusca causes damage by boring, while Fungi cause soft rot. The type of wood used can positively impact its durability, and there is a preference for those that contain high silica content (Dialium sp., Parinari sp, Licania sp, Eschweiler asp., Meirosideros sp.) or contain compounds that act as a repellent (Eusideroxylon zwaggeri, Ocotea rodiaei, Callitris glauca, Eucalyptus marginate). Preservative coatings may also be applied to wood to increase its resistance, and these treatments include Creosote. For removable parts like weir boards, more frequent application of treatments may be needed, or the use of plastics may be considered. In some cases, Polywood® or wood that has a plastic treatment may be used for weir boards.

In summary, there is also a need in the industry to be able to control the harmful impact of predators, competitors, and pests on the aquaculture of aquatic animals without the need for using harmful chemicals, antibiotics or expensive treatment methods. It is thus an object of the present invention to provide a process and equipment that overcome at least some of the problems in prior art.

In some cases, microalgae are co-produced in a single pond system with shrimp (U.S. Pat. No. 3,998,186), fish (U.S. Pat. No. 9,487,716), or shellfish (U.S. Pat. No. 8,753,851 B2). In these synergistic systems, the algae are typically consumed by the shrimp, fish, or shellfish as a source of nutrition. Since these species live in either fresh water, seawater, or brackish water, the salinities of the aqueous growth medium is low enough to sustain life for the shrimp, fish, or shellfish. Thus, these patents and U.S. Pat. No. 6,986,323 discuss synergies between growing algae and aquatic animals at seawater salinities or below, but do not anticipate sequentially growing algae in the same aquaculture pond used to grow the aquatic animals in order to facilitate a treatment to control of microbial, bacterial, viral, predator, competitor, or pest infestations.

### SUMMARY

The present inventors have discovered that by rotating the growth of aquatic animals with algae grown at a higher salinity than that for the aquatic animals within the same aquaculture pond, the microbial (such as pathogenic microbial), bacterial, predator, competitor, and/or pest infestations (hereinafter collectively referred to as “infestations” and “infestation species” for species causing the infestations) that plague the aquatic animal aquaculture systems can be mitigated in an environmentally friendly and economically attractive manner. Accordingly, in one aspect of the present invention, it is now possible to manage the infestations while growing rotating crops of aquatic animals and algae within the same pond. The algae can be used to produce a number of products including, but not limited to algal oil, biofuels, dietary supplements, shrimp food, fish food, poultry food, animal feeds, human foodstuffs, chemical intermediates, and carbon storage.

The growth of aquatic animals and algae with and in the same aquaculture pond provides numerous benefits. First, waste products produced during the growth of aquatic animals can be successfully used for the growth of algae, instead of being discarded, and with minimal transport and storage demands. This reduces material and capitals costs, including reducing costs associated with purchasing, storing, transporting, and delivering nutrients needed for algae growth.

Second, the use of the same aquaculture ponds for both the growth of aquatic animals and algae further saves construction, space, and capital equipment costs to move materials.

Third, as described herein, infestation species (e.g. microbial species) that accumulate in pond bottom solids may cause serious damage to aquatic animal crops over time. As used herein, “pond bottom solids” refers to any sediment materials that accumulate at a bottom of the subject aquaculture pond described herein after at least one cycle of growing aquatic animals in the aquaculture pond.

Since the growth of algae takes place in a second growth medium having a greater salinity than a salinity of a first growth medium in which the growth of aquatic animals takes place, the pond bottom solids may be treated with the greater salinity medium to reduce an amount of infestation species such as pathogenic microbes or density of microbial biomass in the pond bottom solids. Doing so at least reduces time and capital costs associated with sanitizing the ponds and allows the aquaculture ponds to be used for a benefit (i.e. the growth of algae) while maintaining the cleanliness of and suitability of the aquaculture pond for the growth of aquatic animals. For example, the salinity of the second growth medium can be at least about 7 wt-%, at least about 8 wt-%, at least about 9 wt-% or at least about 10 wt-%.

Accordingly, in one aspect, there is disclosed a process for growing both aquatic animals and algae within the same aquaculture pond and/or reducing pathogenic microbes or infestation species in pond bottom solids of the aquaculture pond, wherein the process comprises: growing aquatic animals in the aquaculture pond in a first growth medium; harvesting the aquatic animals from the aquaculture pond; after the harvesting of the aquatic animals, providing a second growth medium in the aquaculture pond, wherein the second growth medium comprises a greater salinity than the salinity of the first growth medium, and the salinity of the second growth medium is at least 7 wt-%; and growing algae in the aquaculture pond in the second growth medium. The process optionally comprises re-introducing an amount of additional first growth medium in the aquaculture pond and re-introducing aquatic animals into the aquaculture pond for further growth of aquatic animals.

According to another aspect, there is disclosed a process for growing aquatic animals and algae and/or reducing infestation species in pond bottom solids, the process comprising growing aquatic animals and algae within the same aquaculture pond in a first growth medium and a second growth medium, respectively, wherein the aquatic animals are optionally harvested before growing algae in the second growth medium, the second growth medium having a greater salinity than the salinity of the first growth medium, the salinity of the second growth medium being optionally at least 7 wt-%.

According to a further aspect, there is disclosed a system for growing both aquatic animals and algae within the same aquaculture pond and/or reducing infestation species in pond bottom solids of the aquaculture pond, the system comprising an aquaculture pond comprising a first growth medium therein for growing the aquatic animals in a first stage and a second growth medium for growing algae in the aquaculture pond in a second stage following the first stage, wherein the second growth medium comprises a greater salinity than the first growth medium.

In one embodiment, the processes further comprise recycling at least a portion of the first growth medium for use in the second growth medium.

In one embodiment, the processes further comprises draining the aquaculture pond after harvesting the aquatic animals and before the growing of algae in the aquaculture pond; and following the draining, filling the drained aquaculture pond with the second growth medium for the growing of the algae.

In one embodiment of the processes, the pond bottom solids of the drained aquaculture pond are allowed to dry, become oxidized, and/or be exposed to UV and/or atmospheric air prior to the filling the drained aquaculture pond with the second growth medium to reduce infestation species or pathogenic microbes in the pond bottom solids.

In one embodiment of the processes, the algae are grown in the aquaculture pond for at least about 24 hours.

In one embodiment, the processes further comprise increasing a salinity of the first growth medium to provide at least a portion of the second growth medium.

In one embodiment, the processes further comprises: after the growing of the algae, directing the second growth medium from the aquaculture pond to one or more additional aquaculture ponds, such as one or more drained additional aquaculture ponds, to produce one or more saline-treated additional aquaculture ponds; reducing a salinity of the second growth medium to generate first growth medium in the one or more saline-treated additional aquaculture ponds; and growing aquatic animals in the first growth medium of the one or more saline-treated additional aquaculture ponds.

In one embodiment, the processes further comprises: removing at least some of the algae and/or the second growth medium from the aquaculture pond after growing algae in the aquaculture pond; after the removing, providing the aquaculture pond with the additional amount of the first growth medium; and optionally re-introducing aquatic animals into the aquaculture pond.

In one embodiment, the processes further comprise: after the growing of algae in the aquaculture pond, discharging at least a portion of the second growth medium to an open body of water as a saline-treated stream.

In one embodiment of the processes, the harvesting of aquatic animals is done by filtering the first growth medium to obtain a retentate comprising the aquatic animals and a filtrate comprising the remaining first growth medium, and wherein the process further comprises: removing the retentate comprising the aquatic animals from the aquaculture pond; increasing a salinity of the filtrate comprising the remaining first growth medium to generate the second growth medium; and directing the second growth medium to the aquaculture pond for the growing of the algae in the aquaculture pond.

In one embodiment, the processes further comprise adding algal nutrients to the second growth medium for promoting the growth of the algae.

In one embodiment, there is disclosed a process for growing aquatic animals and algae and/or reducing pathogenic microbes or infestation species in pond bottom solids, the process comprising growing aquatic animals and algae within the same aquaculture pond in a first growth medium and a second growth medium, respectively, wherein the aquatic animals are harvested before growing algae in the second growth medium, the second growth medium having a greater salinity than the first growth medium, the salinity of the second growth medium being at least 7 wt-%.

In one embodiment, there is disclosed a system for growing both aquatic animals and algae within the same aquaculture pond and/or reducing pathogenic microbes or infestation species in pond bottom solids of the aquaculture pond, the system comprising: an aquaculture pond comprising a first growth medium therein for growing the aquatic animals in a first stage and a second growth medium for growing algae in the aquaculture pond in a second stage following the first stage, wherein the second growth medium comprises a greater salinity than the first growth medium.

In one embodiment, the system further comprises means for harvesting algal, such as an algal harvester for harvesting algae, in fluid communication with the aquaculture pond; and/or means for harvesting aquatic animal, such as an aquatic animal harvester, in fluid communication with the aquaculture pond. In one embodiment, the systems further comprise means for controlling flow, such as a flow controller, disposed within the aquaculture pond, the flow controller being configured to selectively harvest the aquatic animals from the aquaculture pond.

In one embodiment, the systems further comprise one or more additional aquaculture ponds in fluid communication with the aquaculture pond. In the present text, “in fluid communication” denotes that at least liquids can pass, either with or without external help, such as by gravity or using a pump.

In one embodiment of the systems, the one more additional aquaculture ponds comprise at least a portion of the first growth medium for growing aquatic animals and/or at least a portion of the second growth medium for growing algae.

In one embodiment, the systems further comprise means for recycling, such as a recycle conduit, extending directly or indirectly from an outlet of the aquaculture pond to an inlet of the aquaculture pond to recycle at least a portion of the first growth medium or the second growth medium for use in the aquaculture pond. The means for recycling can be any suitable means for transferring the process stream from one end of the pond to another. It may for example be a conduit, a pipe, a gutter, a canal, a channel or similar, or any combination thereof. The recycling means can have means for controlling the flow of the process stream, such as a valve, for example at its inlet (i.e. an outlet of the pond), at its outlet (i.e. an inlet of the pond), in between these two or it may comprise several of such controlling means. If need be, the recycling means can also be equipped with a pump or some other equipment for transferring the process stream. Furthermore, the recycling means can be equipped with means for determining and/or controlling its salinity. For example, a sensor for salinity can be arranged at the outlet or in the beginning of the means for recycling, whereafter the required change in salinity is determined, and the salinity of the recycled material and/or its amount is adjusted accordingly.

In one embodiment, the systems further comprise a source of salinity arranged for providing the second growth medium with a predetermined salinity. The source of salinity can be for example an inlet for adding a liquid having a desired salinity, or an inlet for adding a solid material for increasing salinity.

In one embodiment, the systems further comprise a source of algal nutrients arranged for providing the second growth medium with additional algal nutrients for the growth of the algae. The source of algal nutrients can be for example an inlet for adding a liquid or gas comprising algal nutrients, or an inlet for adding a solid nutrient.

Preferably, the amounts of sources of salinity and/or algal nurients, either solid, gas or liquid, can be controlled with suitable controlling means. The addition can be performed at any suitable location.

In one embodiment, the systems further comprise a source of additional aqueous medium arranged for providing the first growth medium with a predetermined salinity and/or the second growth medium with a predetermined salinity, wherein the additional aqueous medium comprises a member selected from the group consisting of fresh water, seawater, brackish water, and a brine medium having a salinity greater than seawater.

In one embodiment, the systems further comprise one or more aquatic animal harvesters for harvesting aquatic animals and/or one or more algal harvesters for harvesting algae in fluid communication with the aquaculture pond, and means for recycling, such as a recycle conduit arranged for delivering an aqueous medium from an outlet of the one or more algal or aquatic animal harvesters to an inlet of the aquaculture pond, to provide at least a portion of the first growth medium or the second growth medium to the aquaculture pond.

In one embodiment, there is disclosed a system for consecutively growing aquatic animals and algae in the same aquaculture pond comprising: an aquaculture pond; an aquatic animal harvester in fluid communication with the aquaculture pond for growing aquatic animals; and an algal harvester in fluid communication with the aquaculture pond for growing algae.

In one embodiment of the systems and processes, the algae comprises:
one or more microalgal species optionally selected from the group consisting of Amphora sp., Anabaena sp., Anabaena flos-aquae, Ankistrodesmus falcatus, Arthrospira sp., Arthrospira (Spirulina) obliquus, Arthrospira (Spirulina) platensis, Botryococcus braunii, Ceramium sp., Chaetoceros gracilis, Chlamydomonas sp., Chlamydomonas mexicana, Chlamydomonas reinhardtii, Chlorella sp., Chlorella fusca, Chlorella protothecoides, Chlorella pyrenoidosa, Chlorella stigmataphora, Chlorella vulgaris, Chlorella zofingiensis, Chlorococcum citriforme, Chlorococcum littorale, Closterium sp., Coccolithus huxleyi, Cosmarium sp., Crypthecoddinium cohnii, Cryptomonas sp., Cyclotella cryptica, Cyclotella nana, Dunaliella sp., Dunaliella bardawil, Dunaliella salina, Dunaliella tertiolecta, Dunaliella viridis, Euglena gracilis, Fragilaria, Fragilaria sublinearis, Gracilaria, Haematococcus pluvialis, Hantzschia, Isochrysis galbana, Microcystis sp., Monochrysis lutheri, Muriellopsis sp., Nannochloris sp., Nannochloropsis sp., Nannochloropsis salina, Navicula sp., Navicula saprophila, Neochloris oleoabundans, Neospongiococcum gelatinosum, Nitzschia laevis, Nitzschia alba, Nitzschia communis, Nitzschia paleacea, Nitzschia closterium, Nitzschia palea, Nostoc commune, Nostoc flagellaforme, Pavlova gyrens, Peridinium, Phaeodactylum tricornutum, Pleurochrysis carterae, Porphyra sp., Porphyridium aerugineum, Porphyridium cruentum, Prymnesium, Prymnesium paruum, Pseudochoricystis ellipsoidea, Rhodomonas sp., Scenedesmus sp., Scenedesmus braziliensis, Scenedesmus obliquus, Scenedesmus quadricauda, Scenedesmus acutus, Scenedesmus dimorphus, Schizochytrium sp., Scytonema, Skeletonema costatum, Spirogyra, Schiochytrium limacinum, Stichococcus bacillaris, Synechoccus, Tetraselmis sp., Tolypothrix sp., genetically-engineered varieties thereof, and any combinations thereof; or
one or more prokaryotes selected from the group consisting of Aphanothece halophytica, Microcoleus chthonoplastes, M. lyngbyaceus, Spirulina major, S. platensis, Nodularia spumigena, Dactylococcopsis salina, Synechocystis DUN52, PCC 6803, Synechococcus PCC 7418, Phormidium spp., Oscillatoria spp., Lyngbya spp., Halospirulina tapeticola, Microcystis spp., Nostoc spp., and Aphanocapsa spp.; or
one or more eukaryotes selected from the group consisting of Dunaliella spp., Dangeardinella saltitrix, Chlorella vulgaris, Navicula spp., Amphora spp., and Amphora spp.; or
genetically-engineered varieties of any of the above; or
any combinations thereof.

In one embodiment of the systems and processes, the salinity of the second growth medium is at least about 8 wt-%, at least about 9 wt-%, at least about 10 wt-%, at least about 11 wt-%, at least about 12 wt-%, at least about 13 wt-%, at least about 14 wt-%, at least about 15 wt-%, at least about 16 wt-%, at least about 17 wt-%, at least about 18 wt-%, at least about 19 wt-%, at least about 20 wt-%, at least about 21 wt-%, at least about 22 wt-%, at least 23 about wt-%, at least about 24 wt-%, or at least about 25 wt-%, or at least about saturation.

In one embodiment of the systems and processes, the first medium comprises a salinity of from 0 to about 5 wt-%.

In one embodiment of the processes and systems, the difference between the salinities of the first and second growth medium can be e.g. at least 2 wt-%, at least 3 wt-%, at least 4 wt-%, at least 5 wt-%, at least 6 wt-%, at least 7 wt-%, at least 8 wt-%, at least 9 wt-%, at least 10 wt-%, at least 11 wt-%, at least 12 wt-%, at least 13 wt-%, at least 14 wt-%, at least 15 wt-%, at least 16 wt-%, at least 17 wt-%, at least 18 wt-%, at least 19 wt-%, at least 20 wt-%, at least 21 wt-%, at least 22 wt-%, at least 23 wt-%, at least 24 wt-%, or at least 25 wt-%. The difference between the salinities can be for example from at least 2 wt-% up to 25 wt-%, or from at least 2, 3, 4, 5, 6, 7, 8, 9, 10, 11, 12, 13, 14, 15, 16, 17, 18, 19, 20, 21 or 22 wt-% up to 4, 5, 6, 7, 8, 9, 10, 11, 12, 13, 14, 15, 16, 17, 18, 19, 20, 21, 22, 23, 24, or at least 25 wt-%.

In one embodiment of the systems and processes, the aquaculture pond is an open aquaculture pond.

In one embodiment of the systems and processes, the aquatic animals are selected from the group consisting of crustaceans, shrimps, fishes, molluscs, shellfishes, and any combination thereof.

In one embodiment, there is disclosed a system to carry out the process of the present invention or any embodiment of a process as described herein.

In one embodiment, there is disclosed a use of the system as described herein for growing aquatic animals and algae consecutively in the same aquaculture pond, wherein the aquatic animals are harvested before growing algae.

In one embodiment, there is disclosed a process for reducing one or more of the following microbial, microbial pathogen, predator, competitor, pest infestations that are harmful to shrimp, shellfish, crustacean, mollusc, fish aquaculture comprising the steps of a) growing shrimp, shellfish, crustacean, mollusc, and/or fish (e.g. shrimp, shellfish, and/or fish) in an aquaculture pond, b) harvesting or removing the grown shrimp, shellfish, crustacean, mollusc, and/or fish (e.g. shrimp, shellfish, and/or fish) from the aquaculture pond, c) growing algae such as hypersaline algae in the same aquaculture pond, d) removing a substantial portion of the algae (such as hypersaline algae) and algal growth medium from the pond, and e) re-introducing shrimp, shellfish, crustacean, mollusc, and/or fish (e.g. shrimp, shellfish, and/or fish) into the aquaculture pond.

In one embodiment, there is disclosed a process for reducing one or more microbes, microbial pathogens, predators, competitors, and/or pest infestations that are harmful to shrimp, shellfish, fish aquaculture while providing nitrogen and phosphorus nutrients to the algae and halotolerant bacterial comprising the steps of a) growing shrimp, shellfish, fish in an aquaculture pond, b) removing the grown shrimp, shellfish, fish from the aquaculture pond, c) growing hypersaline marine algae in the same aquaculture pond, d) removing a substantial portion of the hypersaline marine algae and algal growth medium from the pond, and e) re-introducing shrimp, shellfish, fish into the aquaculture pond.

In one embodiment, there is provided a process for reducing one or more of the following microbes, microbial pathogens, predators, competitors, pest infestations that are harmful to shellfish aquaculture comprising the steps of a) growing shellfish in an aquaculture pond, b) removing the grown shellfish from the aquaculture pond, c) growing hypersaline marine algae in the same aquaculture pond, d) removing a substantial portion of the hypersaline marine algae and algal growth medium from the pond, and e) re-introducing shellfish into the aquaculture pond.

In one embodiment, there is provided a process for mitigating the presence of pathogenic microbial populations in pond bottom sediment debris comprising the steps of a) growing shrimp in an aquaculture pond, b) removing the grown shrimp from the aquaculture pond, c) growing hypersaline marine algae in the same aquaculture pond, d) removing a substantial portion of the hypersaline marine algae and algal growth medium from the pond, and e) re-introducing shrimp into the aquaculture pond.

In an embodiment, there is disclosed a process for mitigating the presence of pathogenic microbial populations in pond bottom debris comprising the steps of a) growing shrimp in an aquaculture pond, b) removing the grown shrimp from the aquaculture pond, c) growing hypersaline marine algae in the same aquaculture pond, d) removing a substantial portion of the hypersaline marine algae and algal growth medium from the pond, and e) re-introducing shrimp into the aquaculture pond.

In an embodiment, there is disclosed a process for mitigating the presence of pathogenic microbial populations in pond bottom sediment debris comprising the steps: a) growing shrimp in an aquaculture pond, b) removing the grown shrimp from the aquaculture pond, c) growing hypersaline marine algae in the same aquaculture pond, d) removing a substantial portion of the hypersaline marine algae and algal growth medium from the pond, e) re-introducing shrimp into the aquaculture pond and wherein to a drained shrimp-cultivating pond is filled with hypersaline aqueous medium with greater than about 7% salts for cultivating marine algae.

In another embodiment, there is disclosed a process for mitigating the presence of pathogenic microbial populations in pond bottom sediment debris comprising the steps: a) growing shrimp in an aquaculture pond, b) removing the grown shrimp from the aquaculture pond, c) growing hypersaline marine algae in the same aquaculture pond, d) removing a substantial portion of the hypersaline marine algae and algal growth medium from the pond, e) re-introducing shrimp into the aquaculture pond and wherein to a drained shrimp-cultivating pond is filled with hypersaline aqueous medium with a salt concentration of 15 percent up to salt saturation.

In another embodiment, there is disclosed a process for mitigating the presence of pathogenic microbial populations in pond bottom sediment debris, comprising the steps:
a) growing shrimp in a traditional aquaculture pond at salinities of about 1.5-5%,
b) harvesting the market sized shrimp from the aquaculture production pond,
c) growing hypersaline algae in the same pond started with > about 5% salinity media,
d) removing a substantial portion of the algal biomass and the hypersaline growth medium from the pond,
e) re-stocking shrimp into the pond when the salinity of aqueous shrimp culture media can be returned to the about 1.5-5.0% range, and
f) wherein the shrimp-cultivating pond drained for harvest and refilled with hypersaline aqueous medium at a salt concentration of about 15 percent up to saturation.

In an embodiment, there is disclosed a process for mitigating the presence of pathogenic microbial populations in pond bottom sediment debris comprising the steps: a) growing shrimp in an aquaculture pond, b) removing the grown shrimp from the aquaculture pond, c) growing hypersaline marine algae in the same aquaculture pond, d) removing a substantial portion of the hypersaline marine algae and algal growth medium from the pond, e) re-introducing shrimp into the aquaculture pond post salinity balancing, and wherein one or more crops of shrimp are grown prior to growing hypersaline marine algae in the same aquaculture pond.

In another embodiment, there is disclosed a process for reducing bacterial and viruses harmful to shrimp in the shrimp pond debris comprising the steps: a) growing shrimp in an aquaculture pond, b) removing the grown shrimp from the aquaculture pond, c) growing hypersaline marine algae in the same aquaculture pond, d) removing a substantial portion of the hypersaline marine algae and algal growth medium from the pond, e) re-introducing shrimp into the aquaculture pond, and wherein the hypersaline aqueous medium remains in the shrimp pond for more than about 72 hours prior to balancing the pond to a less than about 5% preferred salinity range.

In another embodiment, there is disclosed a process for mitigating the presence of pathogenic microbial populations in pond bottom sediment debris comprising the steps: a) growing shrimp in an aquaculture pond, b) removing the grown shrimp from the aquaculture pond, c) growing hypersaline marine algae in the same aquaculture pond, d) removing a substantial portion of the hypersaline marine algae and algal growth medium from the pond, e) successively transferring the hypersaline media to one or more additional drained shrimp ponds, and f) re-introducing shrimp into a hypersaline treated aquaculture pond(s).

In an embodiment, there is disclosed a process for reducing bacterial and viruses harmful to shrimp in the shrimp pond debris comprising the steps: a) growing shrimp in an aquaculture pond, b) removing the grown shrimp from the aquaculture pond, c) growing hypersaline marine algae in the same aquaculture pond, d) removing a substantial portion of the hypersaline marine algae and algal growth medium from the pond, e) re-introducing shrimp into the aquaculture pond, and wherein the hypersaline aqueous medium charged to the drained shrimp-cultivating pond contains about 15 percent up to salt saturation and wherein said hypersaline aqueous medium remains in the shrimp pond for more than 72 hours prior to balancing the pond to a less than about 5% preferred salinity range.

### BRIEF DESCRIPTION OF THE DRAWINGS

Some of the features and advantages of the disclosure have been stated. Other advantages will become apparent as the description of the disclosure proceeds, taken in conjunction with the accompanying drawings, in which:

FIG. 1 is a schematic diagram of a process in accordance with an aspect.

FIG. 2 is a schematic diagram of a process in accordance with another aspect.

FIG. 3 reveals results of Examples 2-4 after treating samples from aquaculture ponds with aqueous media having different salinities.

### DETAILED DESCRIPTION

As used herein, wt-% refers to a dry mass of a component in a solution in grams divided by 100 grams of the solution. In addition, unless otherwise stated herein or clear from the context, any percentages referred to herein are understood to refer to wt-%.

As used herein, the term “about” refers to a value that is +1% of the stated value. In addition, it is understood that reference to a range of a first value to a second value includes the range of the stated values, e.g., a range of about 1 to about 5 also includes the more precise range of 1 to 5. Further, it is understood that the ranges disclosed herein include any selected subrange within the stated range, e.g., a subrange of about 50 to about 60 is contemplated in a disclosed range of about 1 to about 100.

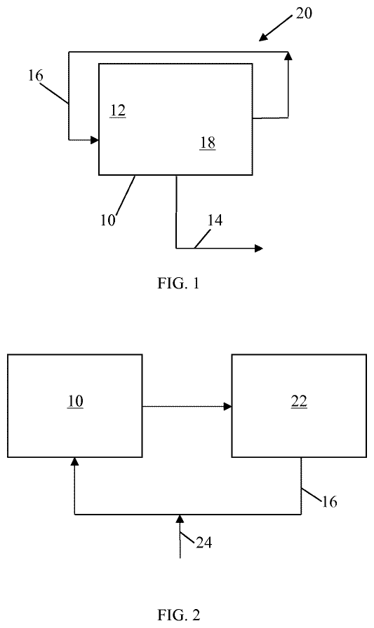

Now turning to the Figures, FIG. 1 illustrates a process and system for carrying out a process in accordance with aspects for reducing one or more of the following microbial, bacterial, viral, predator, competitor, pest infestations by alternating the growth of aquatic animals and algae within the same pond. For ease of discussion, the process will be discussed in detail below, but it is understood that the system includes the structural components needed or desired to carry out the processes described and illustrated herein. First, an aquaculture pond 10 is provided for the growing of aquatic animals or algae therein. The aquaculture pond 10 may be any type of pond used to grow aquatic animals or algae individually known in the art, including, but not limited to enclosed bioreactors (e.g. photobioreactors), open ponds configured either with or without agitation or liners.

In the process, aquatic animals are grown in a first growth medium 12 provided from any suitable source to the aquaculture pond 10. The first growth medium 12 may comprise any suitable components for promoting or enhancing the growth of aquatic animals in the aquaculture pond. In an embodiment, the first growth medium 12 comprises a salinity of from 0 to about 5 wt-%, such as from about 0.5 to about 5 wt-%, for example, 3-5 wt-%. In one embodiment, the first growth medium 12 comprises a salinity of about or less than about 0.5 wt-%, about or less than about 1 wt-%, about or less than about 1.5 wt-%, about 2 wt-%, about or less than about 2.5 wt-%, about 3 wt-%, about or less than about 3.5 wt-%, about or less than about 4 wt-%, or about or less than about 4.5 wt-%.

Following the growth of the aquatic animals in the aquaculture pond 10, the process may include the step of harvesting the aquatic animals from the aquaculture pond 10 to provide an aquatic animal crop (shown as 14). The harvesting may be accomplished by any suitable structure or process, such as by filtering the first growth medium 12 from the aquaculture pond as the first growth medium 12 is discharged from the pond 10. At least a portion of the first growth medium 12 may be directed to one or more additional aquaculture ponds for the growth of aquatic animals or algae therein or may be recycled as a recycled stream 16 from an outlet of the pond to an inlet of the pond (shown by arrow 16 in FIG. 1) as will be discussed in greater detail below.

In a particular embodiment, the harvesting of aquatic animals from the aquaculture pond 10 is done by filtering the first growth medium 12 to obtain a retentate comprising the aquatic animals and a filtrate comprising the remaining first growth medium. The retentate comprising the aquatic animals is removed from the aquaculture pond or other location. Thereafter, the salinity of the filtrate (which comprises first growth medium 12) may be increased to generate an amount of second growth medium 18 by providing an amount of added salts in any suitable form. The second growth medium 18 may then be directed to the aquaculture pond 10 for the growth of algae in the aquaculture pond 10. In one embodiment the filtrate (which comprises first growth medium 12) or a part thereof is discharged from the aquaculture pond and the second growth medium 18 is added to the aquaculture pond or provided in the aquaculture pond.

After the harvesting of the aquatic animals from the aquaculture pond 12, a second growth medium 18 is provided in the aquaculture pond 12. Importantly, the second growth medium 18 comprises a greater salinity than the first growth medium. In certain embodiments, the second growth medium comprises a salinity of at least about 7 wt-%, at least about 8 wt-%, at least about 9 wt-%, at least about 10 wt-%, at least about 11 wt-%, at least about 12 wt-%, at least about 13 wt-%, at least about 14 wt-%, at least about 15 wt-%, at least about 16 wt-%, at least about 17 wt-%, at least about 18 wt-%, at least about 19 wt-%, at least about 20 wt-%, at least about 21 wt-%, at least about 22 wt-%, at least about 23 wt-%, at least about 24 wt-%, or at least about 25 wt-% (e.g. up to saturation). In certain embodiments, the algal aquaculture medium is saturated with salt. In particular embodiments, the salinity of the algal aquaculture medium is from about 7 wt-% to saturation, from about 8 wt-% to saturation, from about 9 wt-% to saturation, from about 10 wt-% to saturation, from about 20 wt-% to saturation, about 10 wt-% to about 25 wt-%, about 10 wt-% to about 20 wt-%, about 10 wt-% to about 15 wt-%, about 12 wt-% to about 25 wt-%, about 15 wt-% to about 25 wt-%, or about 20 wt-% to about 25 wt-%. The salinity of the first growth medium 12 and the second growth medium 18 may comprise any suitable salts for providing the desired salinity. In an embodiment, the salinity comprises sea salts, underground salts, salts of aquifer water, salts of a terminal lake, sodium chloride, and/or any combination of ions present in sea salt.

Thereafter, once the aquaculture pond 10 comprises the second growth medium 18, algae are grown in the aquaculture pond 10 in the second growth medium 18 under suitable conditions for algae growth.

Following growth of the algae in the aquaculture pond 10, the process may comprise re-introducing an amount of additional first growth medium 12 in the aquaculture pond 10 and re-introducing aquatic animals into the aquaculture pond 10 for further growth of aquatic animals. In a particular embodiment, the process comprises removing at least some of the algae and/or the second growth medium 18 from the aquaculture pond after growing algae in the aquaculture pond 10; providing the aquaculture pond 10 with the additional amount of the first growth medium 12; and re-introducing aquatic animals into the aquaculture pond.

Each sequence of an introduction of the first or second growth medium to the aquaculture pond 10 and subsequent growth of aquatic animals or algae therein may be referred to as a “stage.” For example, the providing of the first growth medium 12 in the aquaculture pond 10 and the subsequent growth of aquatic animals in the first growth medium 12 may be referred to as “a first stage” while the providing of the second growth medium 18 in the aquaculture pond 10 and the subsequent growth of algae in the second growth medium 18 may be referred to as a “second stage.” Together, a first stage and a second stage within the same aquaculture pond 10 provide for a “cycle” of aquatic animals and algae growth. The number of cycles of growing aquatic animals and algae in accordance with the present is without limitation. In certain embodiments, the aquaculture pond 10 may be utilized for one, two, three, four, five, ten, twenty, or even more cycles of the growth of aquatic animals and algae within the same pond, e.g., aquaculture pond 10.

In each cycle, the salinity second growth medium 18 for growing algae effectively acts to treat the pond bottom solids and reduce an amount of undesired infestations or pathogenic microbes in the pond bottom solids for a subsequent cycle that begins with growing aquatic animals in the first growth medium 12. As described herein, the “pond bottom solids” may comprise fecal solids, waste feed particles and mineralized residues. Fecal material and byproducts from protein metabolism in the aquatic animal's, e.g., shrimp's, gastrointestinal tract comprises non-digested organic solids, nitrogen, phosphorus, and other micronutrients that are beneficial to algal growth. Aquatic animal feeds and unconsumed feed components typically comprise protein, oils, vitamins, minerals, and other materials. Some of these may be consumed directly by the aquatic animals, while others are consumed by predators, competitors, and pests that in some embodiments may co-exist in the subject pond. The pond bottom solids that accumulate on the bottom of the aquaculture pond 10 may be discharged to any suitable location after being treated/subjected to the salinity of the second growth medium. In certain embodiments, other aquaculture ponds may be discharged to a treatment facility, or released to the ocean. The release of aquaculture pond sediment debris to the ocean without a treatment in accordance with the present invention causes eutrophication and a significant negative environmental impact.

The aquaculture pond 10 may be any type of pond used to grow aquatic animals or algae individually as are known in the art, including, but not limited to enclosed bioreactors (such as photoreactors), open ponds configured either with or without agitation or liners. Unlined ponds comprise earthen borders and pond floors. Suitable liner material can be either plastic or clay. Plastic pond liners are typically formed from polyethylene, polypropylene, or polyvinyl chloride. Different types of these basic polymers can be used, for example linear low-density polyethylene liners are occasionally used for algae cultivation at large scale. These liners may also comprise additives, such as carbon black to provide resistance to ultraviolet radiation. These liners may also comprise Nylon™ or other fibers to provide additional structural integrity. Raven Industries (South Dakota) provides a full line of suitable liners that comprise one or more layers of materials. Suitable clay liners include bentonite clay. However, when ponds are flooded, components in the water can often form a barrier that seals the pond such as cation-exchange-capacity (CEC) of the soil.

It may also be desirable to include liners in just a portion of the pond where it is specifically needed. For example, to protect earthen borders where the hydraulic flow may be elevated. Typically, weir boxes are used for hydraulic flow control in and out of the pond. Weir boxes may be constructed from concrete, wood, high density polyethylene, or other materials or combinations thereof. They may also be fitted with slots to hold screens or barriers to flow. The bottom of the aquaculture ponds typically are designed with less than 2% slope towards the exit of the pond, but that is not essential for the instant invention. In an embodiment, the slope is about 0.5% or more, such as from about 2 to about 3%. Borders that separate one pond from the other are typically of earthen construction, but may comprise rock, concrete, blocks, and other materials to stop the flow of water. Typically, the borders are constructed in such a way that a vehicle may be driven on crown of the border.

The aquaculture pond 10 may be operated in either extensive or intensive mode during shrimp aquaculture. The extensive mode of operating ponds is the traditional operating mode. Aquaculture ponds that are operated in the extensive mode are constructed of earthen borders that are typically unlined. In known processes and systems, seawater is typically used to flush salt from the pond so that the salinity in the pond remains closer to that of seawater. However, this flushing also results in the discharge of some portion of the pond bottom solids into the environment. The water level in extensive ponds is typically less than about one meter. In the intensive mode of operation, the ponds can be lined with a plastic liner, and air may be added in order to mix the ponds and improve oxygen transport. The pond depth in intensive aquaculture typically averages one meter, rarely reaching 1.5 or 2.0 meters in depth. Shrimp stocking densities in hyper intensive aquaculture systems can be about ten to twenty times greater than traditional earthen pond systems operated under the extensive management protocols.

The first growth medium 12 and the second growth medium 18 may be provided with their respective desired salinities by any suitable process. In an embodiment, the desired salinity is achieved by evaporating seawater to concentrate the sea salts therein. In other embodiments, other sources of salt may be used, such as from desalination blowdown, saline aquafers, terminal lakes, and mined sodium chloride. Higher concentrations of NaCl typically slow the growth rate of algae but can be used to select specific algal species. For example, Dunaliella salina can still survive at elevated salinities, while other algae cannot.

In addition to different salinity, the aquaculture pond 10 may be operated differently in other aspects beyond the salinity of the growth medium used. In particular, additional differences may include pond depth, mean residence time, batch versus continuous flow operation, nutrient addition, and/or fresh water addition, and combinations thereof.

In certain embodiments, the mean residence time for the growth of algae in the aquaculture pond may range from about half a day, about one day or about two days to two weeks or more, in part depending on the algae growth rate and the time required to treat the pond bottom solids for infestations such as pathogenic microbes that are harmful to aquatic animals. In a particular embodiment, the mean residence time is at least about 12 hours or at least about 24 hours for the growth of algae in the aquaculture pond 10. The residence time used for keeping the aquaculture pond filled with the second growth medium for the algae can range from about half a day, one or more days to years, depending upon the desired goals. There are several potential goals that may be achieved by filling the aquaculture pond 10 with the second growth medium 18 and growing algae in the pond 18:1) to minimize time between aquatic animal crops; and 2) to utilize time between aquatic animal, e.g., shrimp, harvest in the fall and the time when aquatic animals may be re-introduced the following year; and 3) to utilize the pond 10 for one or more seasons after the aquaculture pond contained aquatic animals.

As described herein, the pond bottom solids may comprise fecal solids, waste feed particles and mineralized residues. Fecal material and byproducts from protein metabolism in the aquatic animals', e.g., shrimp's, gastrointestinal tract comprises non-digested organic solids, nitrogen, phosphorus, and other micronutrients that are beneficial to algal growth. Aquatic animal feeds and unconsumed feed components typically comprise protein, oils, vitamins, minerals, and other materials. Some of these may be consumed directly by the aquatic animals, while others are consumed by predators, competitors, and pests that co-exist in the subject pond. The pond bottom solids that accumulate on the bottom of the aquaculture pond, may be discharged to any suitable location after being treated/subjected to the salinity of the second growth medium. In certain embodiments, other aquaculture ponds may be discharged to a treatment facility, or released to the ocean. The release of shrimp pond sediment debris to the ocean causes eutrophication and is a significant negative environmental impact.

As mentioned previously, in one aspect, waste nutrients from the aquaculture pond 10 may be a source of nutrients to the algal aquaculture. By way of example, the average chemical profile of nutrient concentrated wastewater effluent discharged from high-density shrimp aquaculture systems has been analyzed and demonstrated to be composed of the following parameters: total nitrogen +/−260 mg/liter, ammonia nitrogen +/−46 mg/liter, nitrite nitrogen +/−0.06 mg/liter, nitrate nitrogen +/−126 mg/liter, total phosphorus +/−173 mg/liter, phosphate phosphorous +/−40 mg/liter, biological oxygen demand +/−1350 mg/liter, chemical oxygen demand +/−3740 mg/liter, and total volatile solids >7,000 mg/liter. These waste nutrients may remain in the pond 12 after the growth of aquatic animals and/or are carried via the first growth medium and at least a portion of the first growth medium is used for the second growth medium.

In certain embodiments, supplemental algal nutrients may be added to the aquaculture pond 10 or otherwise provided in the second growth medium 18 for the growth of algae from suitable source(s) thereof. The waste nutrients and/or the supplemental nutrients may comprise nitrogen, phosphorus, iron, trace mineral nutrients, and combinations thereof. Suitable nitrogen sources include, but are not limited to ammonia, urea, nitrates, or combinations thereof. Suitable phosphorus sources include, but are not limited to phosphoric acid, diammonium phosphate, phosphates, and other sources of phosphorus. Suitable iron sources are EDTA chelated iron, and other soluble and insoluble forms of iron. There are a number of other micronutrients that are needed by algae, such as sulfur and manganese, copper, zinc, molybdenum and boron that can be added as supplemental nutrients. Many of these micronutrients are contained in seawater and other sources of water.

Following the growth of algae, at least a portion of the second growth medium 18 may be removed from the aquaculture pond 10. In certain embodiments, the (used) second growth medium 18 may be charged to another aquaculture pond, discharged to an appropriate body of water, such as the ocean, utilized for solar salt production, recycled, or a combination thereof.

The processes and systems disclosed herein are further able to reduce capital and material costs by enabling reuse of growth medium from the growth of aquatic animals for the growth of algae. Accordingly, in one embodiment, the process may further comprise recycling at least a portion of the first growth medium 12 for use in the second growth medium 18 as was shown by arrow 16 in FIG. 1. Since the second growth medium will have a greater salinity than the first growth medium, it is contemplated a source of salt in solid or liquid form may be added to the recycled first growth medium and/or the first growth medium will be added to a medium having a greater salinity to form the second growth medium. Thus, in certain embodiments, the process may further comprise increasing a salinity of the first growth medium to provide at least a portion of the second growth medium.

In certain embodiments, the aquatic animals may be removed from the pond 10 and a portion, but not all, of the first growth medium 12 is discharged from and recycled to the aquaculture pond 10. In other embodiments, the contents of the pond 10 are completely drained prior to the growing of algae in the aquaculture pond. In a particular embodiment, the process thus comprises draining the aquaculture pond 10 after harvesting the aquatic animals and before the growing of algae in the aquaculture pond 10; and following the draining, filling the drained aquaculture pond 10 with the second growth medium for the growing of the algae.

In certain embodiments, the treatment of the pond bottom solids by the second growth medium 18 may be supplemented with an additional treatment method, particularly when the aquaculture pond 10 is drained since the pond bottom solids are then exposed to the atmosphere. In an embodiment, once the first growth medium 12 or the second growth medium 18 are drained from the aquaculture pond 10, the pond bottom solids of the drained aquaculture pond 10 are allowed to dry, become oxidized, and/or be exposed to UV and/or atmospheric air prior to the filling the drained aquaculture pond 10 with the second growth medium 18 to reduce infestations in the pond bottom solids.

It is contemplated that in one aspect the second growth medium 18 may advantageously be utilized downstream of the aquaculture pond 10 in which both the growth of aquatic animals and algae takes place to maximize the utilization of materials in the processes and systems.

In an embodiment, as shown in FIG. 2, the second growth medium 18 may be utilized not only to treat pond bottom solids of the aquaculture pond 10, but also additional aquaculture ponds. Accordingly, in an embodiment, after the growing of the algae in the aquaculture pond 10, the process may further comprise directing the second growth medium 18 from the aquaculture pond 10 to one or more additional aquaculture ponds 22 to produce one or more saline-treated additional aquaculture ponds. The additional aquaculture pond(s) 22 may then be utilized to grow additional aquatic animals and/or algae. In an embodiment, the additional aquaculture pond(s) are utilized to grow aquatic animals.

Further, in certain embodiments, the second growth medium 18 may be discharged from the aquaculture pond 10 to at least one additional aquaculture pond 22 in fluid communication therewith. There, the second growth medium 18 may be provided with a sufficient residence time, e.g., about 2 hours to about 48 hour or more, in the additional aquaculture pond(s) 22 to thus provide one or more saline-treated additional aquaculture pond(s). In certain embodiments, further algae is grown in the saline-treated additional aquaculture pond(s).

In an embodiment, the process may further include removing at least a portion of the second growth medium 18 from the saline-treated additional aquaculture pond(s) after the treating of the additional aquaculture pond(s) 22 with the second growth medium 18 and/or growing algae; and providing the saline-treated additional aquaculture pond(s) with the first growth medium 12 and growing aquatic animals therein.

In certain embodiments, the first growth medium 12 may be provided in the saline-treated additional aquaculture pond(s) by reducing a salinity of the second growth medium 18 to generate the first growth medium 12 in the saline-treated additional aquaculture pond(s). This may be accomplished by combining the second growth medium 18 with a suitable amount of an aqueous medium (shown by arrow 24) that reduces the salinity of the first growth medium 12 to the desired degree.

In an embodiment, after the growing of algae in the aquaculture pond, the process may comprise discharging at least a portion of the second growth medium 18 from the aquaculture pond 10 or any additional aquaculture ponds to an open body of water as a saline-treated stream. As discussed previously, the greater salinity of the second growth medium 18 is effective to at least reduce an amount of infestation species in the pond bottom solids before discharge, thereby resulting in a much more environmentally friendly discharge stream relative to known processes.

In another aspect, there are disclosed systems for growing both aquatic animals and algae within the same aquaculture pond and/or reducing infestation species in pond bottom solids of the aquaculture pond. In an embodiment and referring again to FIG. 1, there is shown a system 20 comprising the aquaculture pond 10 that comprises the first growth medium 12 therein for growing aquatic animals in a first stage and the second growth medium 18 for growing algae in the aquaculture pond in a second stage following the first stage. In the system, the second growth medium 18 comprises a greater salinity than the first growth medium. In certain embodiments, the first growth medium 12 comprises a salinity of from 0 to about 5 wt-%, such as from about 0.5 to 5 wt-%, e.g. about 3 to about 5 wt-%, about or less than about 0.5 wt-%, about or less than about 1 wt-%, about or less than about 1.5 wt, about or less than about 2 wt-%, about or less than about 2.5 wt-%, about or less than about 3 wt-%, about or less than about 3.5 wt-%, about or less than about 4 wt-% or about or less than about 4.5 wt-%. The second growth medium 18 comprises a salinity of at least about 7 wt-%, at least about 8 wt-%, at least about 9 wt-%, at least about 10 wt-% or more as described herein.

In certain embodiments, the system further comprises one or more additional aquaculture ponds in fluid communication with the aquaculture pond. In an embodiment, the additional aquaculture pond(s) comprise at least a portion of the first growth medium 12 for growing aquatic animals and/or at least a portion of the second growth medium 18 for growing algae.

In an embodiment, the system 20 may further comprise means for harvesting algae, such as one or more algal harvesters for harvesting algae, in fluid communication with the aquaculture pond 10. In this way, algae can be harvested in the system 20 utilizing the first growth medium 12 or the second growth medium 18.

In an embodiment, means for controlling flow, such as one or more flow controllers, can be disposed at any suitable location in the system 20 to control the movement of materials therein. In a particular embodiment, the system 20 comprises a flow controller within the aquaculture pond 10 or within a conduit extending from any additional components in the system to and from the aquaculture pond 10. In an embodiment, the flow controller may comprise a weir, wherein the weir can be configured to act as a filter to selectively harvest aquatic animals from the aquaculture pond 10 and optionally deliver the first growth medium 12 that passes through as a recycle stream back to the aquaculture pond 10 or downstream to an additional aquaculture pond or a downstream harvester.

In an embodiment, the system may comprise any suitable structure for delivering materials through the system. In an embodiment, the first growth medium 12 or the second growth medium 18 may be directed through a recycle conduit extending directly or indirectly from an outlet of the aquaculture pond 10 to an inlet of the aquaculture pond to recycle at least a portion of the first growth medium 12 or the second growth medium 18 for use in the aquaculture pond 10. In certain embodiments, the recycle conduit extends directly from an outlet to an inlet of the aquaculture pond 10 as was shown in FIG. 1.

In other embodiments, the recycle conduit extends indirectly from an outlet of the pond to an inlet of the aquaculture pond 10. In a particular embodiment, the system further comprises one or more aquatic animal harvesters for harvesting aquatic animals and/or one or more algal harvesters for harvesting algae in fluid communication with the aquaculture pond, and optionally a recycle conduit is arranged for delivering an aqueous medium from an outlet of the one or more algal or aquatic animal harvesters to an inlet of the aquaculture pond to provide at least a portion of the first growth medium or the second growth medium to the aquaculture pond 10. The salinity, nutrient, or any other parameter may be adjusted by modifying the subject medium within the pond or prior to being directed to the aquaculture pond 10.

It is further contemplated that any parameter of the first or second growth medium may be modified within the aquaculture pond 10 or prior to being delivered to the aquaculture pond 10. In an embodiment, the parameter comprises a salinity, nutrient content, or pH. The system may thus naturally comprise any suitable source for adjusting the parameter, e.g., salinity, nutrient, or pH. In an embodiment, the system further comprises a source of salinity arranged for providing the first or second growth medium 12, 18 with a predetermined salinity. In an embodiment, the source of salinity is in fluid communication with the recycle conduit for increasing a salinity of the first growth medium 12 or another aqueous medium utilized to generate the second growth medium 18.

In other embodiments, the system may further comprise a source of algal nutrients arranged for providing the second growth medium 18 with additional algal nutrients for the growth of the algae. In still other embodiments, the system may further comprise a suitable source of nutrients for providing the first growth medium 12 with nutrients for the growth of aquatic animals.

In certain embodiments, the salinity of the second growth medium or other growth medium may be reduced. In such instances, the system further comprises a source of additional aqueous medium arranged for providing the first growth medium with a predetermined salinity. In such embodiments, the additional aqueous medium comprises a member selected from the group consisting of fresh water, seawater, brackish water, and a brine medium having a salinity greater than seawater.

In one embodiment, the systems disclosed and encompassed by the scope of the present application herein are configured to carry out a process as is disclosed and encompassed by the scope of the present application.

In other embodiments, the systems disclosed and encompassed by the scope of the present application are used to grow aquatic animals and algae consecutively in the same aquaculture pond.

In one embodiment of the processes, systems or uses, the algae are selected from the group comprising or consisting of, or the algae comprise:
one or more microalgae, optionally microalgal species selected from the group consisting of Amphora sp., Anabaena sp., Anabaena flos-aquae, Ankistrodesmus falcatus, Arthrospira sp., Arthrospira (Spirulina) obliquus, Arthrospira (Spirulina) platensis, Botryococcus braunii, Ceramium sp., Chaetoceros gracilis, Chlamydomonas sp., Chlamydomonas mexicana, Chlamydomonas reinhardtii, Chlorella sp., Chlorella fusca, Chlorella protothecoides, Chlorella pyrenoidosa, Chlorella stigmataphora, Chlorella vulgaris, Chlorella zofingiensis, Chlorococcum citriforme, Chlorococcum littorale, Closterium sp., Coccolithus huxleyi, Cosmarium sp., Crypthecoddinium cohnii, Cryptomonas sp., Cyclotella cryptica, Cyclotella nana, Dunaliella sp., Dunaliella bardawil, Dunaliella salina, Dunaliella tertiolecta, Dunaliella viridis, Euglena gracilis, Fragilaria, Fragilaria sublinearis, Gracilaria, Haematococcus pluvialis, Hantzschia, Isochrysis galbana, Microcystis sp., Monochrysis lutheri, Muriellopsis sp., Nannochloris sp., Nannochloropsis sp., Nannochloropsis salina, Navicula sp., Navicula saprophila, Neochloris oleoabundans, Neospongiococcum gelatinosum, Nitzschia laevis, Nitzschia alba, Nitzschia communis, Nitzschia paleacea, Nitzschia closterium, Nitzschia palea, Nostoc commune, Nostoc flagellaforme, Pavlova gyrens, Peridinium, Phaeodactylum tricornutum, Pleurochrysis carterae, Porphyra sp., Porphyridium aerugineum, Porphyridium cruentum, Prymnesium, Prymnesium paruum, Pseudochoricystis ellipsoidea, Rhodomonas sp., Scenedesmus sp., Scenedesmus braziliensis, Scenedesmus obliquus, Scenedesmus quadricauda, Scenedesmus acutus, Scenedesmus dimorphus, Schizochytrium sp., Scytonema, Skeletonema costatum, Spirogyra, Schiochytrium bacillaris, Synechoccus, Tetraselmis sp., limacinum, Stichococcus Tolypothrix sp., genetically-engineered varieties thereof, and any combinations thereof; or
one or more prokaryotes selected from the group consisting of Aphanothece halophytica, Microcoleus chthonoplastes, M. lyngbyaceus, Spirulina major, S. platensis, Nodularia spumigena, Dactylococcopsis salina, Synechocystis DUN52, PCC 6803, Synechococcus PCC 7418, Phormidium spp., Oscillatoria spp., Lyngbya spp., Halospirulina tapeticola, Microcystis spp., Nostoc spp., and Aphanocapsa spp.; or
one or more eukaryotes selected from the group consisting of Dunaliella spp., Dangeardinella saltitrix, Chlorella vulgaris, Navicula spp., and Amphora spp.; or
genetically-engineered varieties of any of the above; or
any combinations thereof.

In one embodiment, the algae or microalgae have not been genetically modified or do not originate from genetically engineered algae or microalgae. In a specific embodiment, the algae or microalgae is selected from the group comprising or consisting of Dunaliella sp., Dunaliella bardawil, Dunaliella salina, Dunaliella tertiolecta, Dunaliella parva and Dunaliella viridis, and any combination thereof. In a specific embodiment, the algae or microalgae is Dunaliella salina.

In one embodiment of the processes, systems or uses described herein, the aquaculture pond and/or additional aquaculture pond(s) comprise or are an open aquaculture pond/open aquaculture ponds.

In one embodiment of the processes, systems, or uses, the size of the aquaculture pond is about 0.1—about 1000 hectares, about 0.1—about 200 hectares, about 0.1—about 100 hectares, about 0.1—about 20 hectares, about 1—about 50 hectares, about 1—about 20 hectares, about 1—about 10 hectares, about 1—about 5 hectares, or less than about 1 hectare or about 0.1 hectare.

In one embodiment of the processes and systems described herein, the aquatic animals are selected from the group consisting of crustaceans, shrimps, fishes, molluscs, shellfishes, and any combination thereof.

The processes and systems described herein may be utilized to reduce and/or eliminate a multitude of infestation species from pond bottom solids of the ponds described herein. These infestation species may include but are not limited to the following microbes (such as pathogenic microbes), bacteria, viruses, predators, competitors, and pests referred to above in the Background section and described below.

Predators from ponds that are reduced in concentration or eliminated entirely by the hypersaline (second growth) medium include, but are not limited to fish and/or crabs.

Competitors from ponds that are reduced in concentration or eliminated entirely by the hypersaline media include, but are not limited to snails, fish, crabs, and shrimp.

Pests that live in ponds that are reduced in concentration or eliminated entirely by the hypersaline medium include but are not limited to burrowing shrimp (Thalassina), Mud worm egg cases, organisms that degrade wood, shells, and crabs.

Suitable hypersaline algae (such as hypersaline marine algae) include, but are not limited to the Prokaryotes Aphanothece halophytica (aka Coccochloris elabens, Cyanothece, Halothece), Microcoleus chthonoplastes; M. lyngbyaceus, Spirulina major; S. platensis, Nodularia spumigena, Dactylococcopsis salina, Synechocystis DUN52, and PCC 6803, Synechococcus PCC 7418, Phormidium spp. (e.g. P. ambiguum, P. tenue), Oscillatoria spp. (e.g. O. neglecta, O. limnetica, O. salina), Lyngbya spp. (e.g. L. majuscula, L. aestuarii), Halospirulina tapeticola, Microcystis spp., Nostoc spp., Aphanocapsa spp., and the Eukaryotes Dunaliella spp. (D. salina, D. viridis, D. parva, etc.), Dangeardinella saltitrix, Chlorella vulgaris, Navicula spp., Amphora spp. Amphora spp. and combination thereof. Characteristics of some hypersaline marine algae (hypersaline microalgae) are described in Table 4.

TABLE 4
List of Hypersaline Microalgae

| | Characteristics and Salinity | |
|---|---|---|
| Microalgae | Tolerance | Structure |
| Prokaryotes - | | |
| Cyanobacteria | | |
| Aphanothece halophytica | Optimally at 16-23%; can be | Unicellular with cell |
| (aka Coccochloris elabens, | found at saturation; benthic | wall |
| Cyanothece, Halothece) | mat growth | |
| Microcoleus | Benthic mat-forming; salinity | Sheathed filament |
| chthonoplastes; M. | up to 20% and higher; found | |
| lyngbyaceus | in salterns | |
| Spirulina major; S. | Found in Great Salt Lake | Filament |
| platensis | (GSL) and salterns; mat- | |
| | forming | |
| Nodularia spumigena | Found in GSL | Filament |
| Dactylococcopsis salina | Found in salterns with high | |
| | sulfide concentrations; 5-20% | |
| | salinity | |
| Synechocystis DUN52, and | S. DUN52 up to saturation; S. | Unicellular |
| PCC 6803 | PCC 6803 up to 8.75 to 10.5% | |
| Synechococcus PCC 7418 | | Unicellular |
| Phormidium spp. (e.g. P. | Found in GSL up to 18% | Filament |
| ambiguum, P. tenue) | | |
| Oscillatoria spp. (e.g. O. | Found in GSL and salterns; | Filament |
| neglecta, O. limnetica, O. | mat-forming | |
| salina) | | |
| Lyngbya spp. (e.g. L. | Found in salterns; mat-forming | Filament |
| majuscula, L. aestuarii) | | |
| Halospirulina tapeticola | | Coiled Filament |
| Microcystis spp. | Up to 18% | |
| Nostoc spp. | Up to 18% | |
| Aphanocapsa spp. | 6 to 36% | Small cells singly or in |
| | | pairs at low salinity; |
| | | large rounded |
| | | vacuolated cells at high |
| | | salinity |
| Eukaryotes | | |
| Dunaliella spp. (D. salina, | Optimally at 7-20%; up to | Cell membrane; |
| D. viridis, D. parva, etc.) | saturation | unicellular green alga |
| Dangeardinella saltitrix | Up to saturation | Cell membrane; |
| | | unicellular green alga |
| Chlorella vulgaris | Hypersaline lakes, 3-25% | Unicellular with cell |
| | | wall |
| Navicula spp. | Up to 21% salinity | Diatom |
| Amphora spp. | Up to 21% salinity | Diatom |
| Isochrysis spp. | Up to 10% salinity | Cell membrane; |
| | | unicellular green alga |

Aqueous medium of hypersalinity (about 7% or greater salts) is effective in destroying via changing osmotic pressure bacteria that are acclimated to sea water salinity (3.5% salts by weight). When such bacteria are in a hypersaline solution, the concentration of water in the hypersaline solution is less than that inside the bacterial cell. Because of the osmotic pressure difference, water tends to leave the cell. This causes the cell to dehydrate and the process eventually kills the bacteria.

Proteins are complex organic macromolecules that contain carbon, hydrogen, oxygen, nitrogen, and usually sulfur and are composed of one or more chains of amino acids. Proteins are fundamental components of all living cells and include many substances, such as enzymes, hormones, and antibodies that are necessary for the proper functioning of an organism.

Viruses are DNA or RNA encased in protein. Viruses can be classified as naked or enveloped. The naked viruses have their DNA or RNA surrounded by a simple protein coating. Enveloped viruses are surrounded by phospholipids that they steal from the cells that they parasitize. Enveloped viruses can be rendered harmless when their viral envelope is destroyed, because the virus no longer has the recognition sites necessary to identify and attach to host cells. Enveloped viruses have protein probes projecting through their phospholipid coating.

Protein denaturation occurs because the bonding interactions responsible for the secondary structure (hydrogen bonds to amides) and tertiary structure are disrupted. In tertiary structure there are four types of bonding interactions between “side chains” including: hydrogen bonding, salt bridges, disulfide bonds, and non-polar hydrophobic interactions that may be disrupted. Therefore, a variety of reagents and conditions can cause denaturation by application of some external stress or compound, such as a strong acid or base, a concentrated inorganic salt, an organic solvent (e.g., alcohol or chloroform), or heat. If proteins in a living cell are denatured, this results in disruption of cell activity and possibly cell death.

A chaotropic agent is a substance which disrupts the three dimensional structure in macromolecule such as protein, DNA (Deoxyribonucleic acid) or RNA (Ribonucleic acid) and denatures them. Chaotropic agents interfere with stabilizing intramolecular interactions mediated by non-covalent forces such as hydrogen bonds and van der Waals forces.

Known chaotropic reagents include Urea at 6-8 molarity, Thiourea at a molarity of 2, Guanidiniium chloride at 6 molarity and Lithium Perchlorate of 4.5 molarity. The reagents are expensive and have a variety of other undesirable characteristics that render them unsuitable for discharge into the environment.

The present inventors have discovered that a hypersaline aqueous medium, concentrated (at least about 7% by weight, such as at least about 10% by weight) solution of sea salts are effective both in killing bacteria and deactivating viruses present in the shrimp pond waste. Although we do not wish to be bound by our theory, it is believed that bacteria are inactivated due to the extreme change in osmotic pressure in going from a salt concentration of around 3.5% used in shrimp farming to hypersaline solutions. Likewise, in concentrated solution of sea salts, the protein coating of viruses that protect their RNA or DNA are rendered inactive by the highly saline solution of the algal aquaculture facility denaturing their protein coating.

Viral diseases of cultured aquatic animals that are deactivated (killed) by the present processes and systems include, but are not limited to, the DNA viruses Monodon baculovirus, Baculoviral midgut gland necrosis virus, White spot syndrome virus, Infectious hypodermal and haematopoletic necrosis virus and the Hepatopancreatic parvovirus.

RNA viruses include, but are not limited to, Yellow head virus, Taura syndrome virus, Macrobrachium rosenbergil nodavirus (White Tail Disease), Laem-Singh virus and Mourllyan virus.

Bacteria inactivated by the procedures include: Vibriosis, Necrotizing Hepatopancreatitis, Zoea II Syndrome, Mycobacteriosis and Rickettsial Disease.

Vibriosis is also known as Blackshell Disease, Septic Hepatopancreatic Necrosis, Tail Rot, Brown Gill Disease, Swollen Hindgut Syndrome, Firefly Disease and Luminous Bacterial Disease.

Necrotizing Hepatopancreatitis, NHP, also known as Texas Necrotizing Hepatopancreatitis (TNHP), Granulamatous hepatopancreatitis, Texas Pond Mortality Syndrome (TPMS), Peru Necrotizing Hepatopancreatitis (PNHP is a severe bacterial disease affecting penacid shrimp aquaculture. NHP results in significant mortalities and devastating losses to shrimp crops. Elevated salinity and temperature above that in typical shrimp aquaculture appear to be stress factors for the shrimp and are associated with NHP outbreaks. The magnitude of the elevated salinities in shrimp aquaculture are just slightly elevated over seawater, with values of about 4 to 5 wt-%. Zoea II Syndrome has no known treatment.

Mycobacteriosis, also known as Mycobacterium Infection of Shrimp and Shrimp Tuberculosis has no other proven treatment but prolonged use of a combination of antimicrobials is thought to be effective.

Rickettsial Disease has no proven treatment.

Prior to startup of the hypersaline media treatment of the drained shrimp pond, the pond bottom solids may optionally be tilled to expose lower layers of the debris to the second growth medium.

There is increasing interest in using algal biomass for a plethora of sustainable activities, such as a source of renewable energy, as a mode to safely and efficiently capture carbon dioxide from the atmosphere for carbon sequestration, and as a renewable source of chemical intermediates.

From a sustainability perspective, algal strains of commercial interest preferably do not utilize fresh water in their growth process, but use water derived from the ocean or saline aquifers to offset water losses due to evaporation from the open ponds. This constraint, based on sustainability, favors the use of algae (such as marine algae) that live in a saline to hypersaline growth medium. The salt content, by weight, of a hypersaline medium can be as much as 18 times saltier than the large oceans, which usually have a salinity level of 3.2 to 3.5%.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. That said, it is understood that any one or more features disclosed herein may be combined.

As used herein, a plurality of items, structural elements, compositional elements, and/or materials may be presented in a common list for convenience. However, these lists should be construed as though each member of the list is individually identified as a separate and unique member. Thus, no individual member of such a list should be construed as a de facto equivalent of any other member of the same list solely based on their presentation in a common group without indications to the contrary. In addition, various embodiments and examples of the present invention may be referred to herein along with alternatives for the various components thereof. It is understood that such embodiments, examples, and alternatives are not to be construed as de facto equivalents of one another but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

### EXAMPLES

### Example 1

A 100-hectare traditional shrimp aquaculture pond system in Sinaloa Mexico was used to grow shrimp for more than 10 years, but the yield (farm productivity) had begun to decline significantly due to the impact of microbial disease. Spores of the vibrio bacteria can stay in the soil and be transferred between cultures from year to year. In order to remediate the shrimp aquaculture pond system, the ponds were completely drained, and the bottom of the ponds were tilled in order to liberate nutrients and make them available to algae. Ponds in the shrimp aquaculture pond system were then flooded with 300 micron filtered seawater and water was allowed to evaporate until the salinity reached about 20 wt-% NaCl. At this salinity, certain species of Dunaliella survived and thrived by using the shrimp pond debris as a nitrogen and phosphorus nutrient source. This algal aquaculture medium was transferred throughout the pond system to treat the ponds by exposing them to the hypersaline conditions in the fall. After the pond system was treated, the ponds were flushed with seawater to rinse out any excess salt. Then, shrimp larvae were introduced to start the new shrimp production cycle in April of the following year. These shrimp ponds performed at commercial level for the next five years.

### Example 2

A marine sediment sample was collected from Pond 1 in a traditional shrimp aquaculture farm located in Sonora, Mexico. The sample comprised wet, black mud from the bottom sediments of the shrimp pond that contained organic matter. The 20 liter sample was collected and immediately placed on ice until subsamples of the marine sample were withdrawn the following day. Test tubes were prepared with salinity increments of 0, 2, 8, 12, 18, and 25 wt-% NaCl by using a saturated solution of marine brine and diluting with fresh water. A total of nine milliliters of each of these salinity increments were placed in a test tube and 1.0 grams of sediment was added to each test tube at the different salinity increments. The test tubes were placed in an orbital incubator operating at 150 rpm for 24 hours and at 30 degrees Celsius. Thereafter, 100 microliter samples were seeded onto Thiosulfate Citrate Bile Sucrose agar plates (TCBS) by extension, and incubated for 24 hours at 30 degrees Celsius.

After this incubation period, the Colony Forming Unit (CFUs) per gram, or CFU's/gram, were counted. The Vibrio bacteria incubated on TCBS agar produce either yellow or green colonies, depending if they could ferment sucrose, or not. When sucrose fermentation occurs, yellow colonies are produced by species, such as Vibrio cholera. Vibrio species known to be shrimp pathogens such as Vibrio parahaemolyticus, produce green colonies when incubated on TCBS agar. In all of the colonies observed, about 95% of the colonies during analysis were yellow, and about 5% of the colonies were green. The kill rate at the different salinities was based on the CFU/gram count counted at 2 wt-% NaCl. The kill rate was computed as: (1-(CFU/gram at the salinity of interest divided by the CFU/gram at 2 wt-% NaCl))×100%. At 8 and 12 wt-% NaCl, the percentage reduction in CFU's/gram were 91.3 and 98.9%, respectively. No CFU's/gram were observed at 18 and 25 wt-% NaCl-thus the percentage reduction in CFU's/gram were essentially 100%, within measurement accuracy.

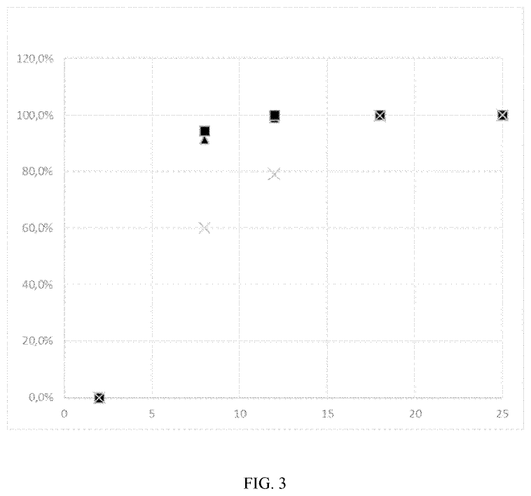

A graphical representation of the results is shown in FIG. 3, with triangles, the treatment salinity (wt-% NaCl) on the x-axis and counts/counts at 2 wt-% NaCl on the y-axis. Excellent reduction of Vibrio species was shown already at 8 wt-% NaCl and essentially no Vibrio species were found at salinities at or above 12 wt-% NaCl after treatment for 24 hours.

### Example 3

A marine sediment sample was collected from Pond 2 in a traditional shrimp aquaculture farm located in Sonora, Mexico. The sample comprised wet, black mud from the bottom sediments of the shrimp pond that contained organic matter. The 20 liter sample was collected and immediately placed on ice until subsamples of the marine sample were withdrawn the following day. Test tubes were prepared with salinity increments of 0, 2, 8, 12, 18, and 25 wt-% NaCl by using a saturated solution of marine brine and diluting with fresh water. A total of nine milliliters of each of these salinity increments were placed in a test tube and 1.0 grams of sediment was added to each test tube at the different salinity increments. The test tubes were placed in an orbital incubator operating at 150 rpm for 24 hours and at 30 degrees Celsius.

Thereafter, 100 microliter samples were seeded onto Thiosulfate Citrate Bile Sucrose agar plates (TCBS) by extension, and incubated for 24 hours at 30 degrees Celsius. After this incubation period, the Colony Forming Unit (CFUs) per gram, or CFU's/gram, were counted. The Vibrio bacteria incubated on TCBS agar produce either yellow or green colonies, depending if they can ferment sucrose, or not. When sucrose fermentation occurs, yellow colonies are produced by species, such as Vibrio cholera. Vibrio species known to be shrimp pathogens such as Vibrio parahaemolyticus, produce green colonies when incubated on TCBS agar. In all of the colonies observed, about 95% of the colonies during analysis were yellow, and about 5% of the colonies were green. The kill rate at the different salinities was based on the CFU/gram count counted at 2 wt-% NaCl. The kill rate was computed as: (1-(CFU/gram at the salinity of interest divided by the CFU/gram at 2 wt-% NaCl))×100%. At 8 wt-% NaCl, the percentage reduction in CFU's/gram was 94.4%. No CFU's/gram were observed at 12, 18, and 2 5 wt-% NaCl
thus the percentage reduction in CFU's/gram were essentially 100%, within measurement accuracy at these salinities.

A graphical representation of the results is shown in FIG. 3, with squares, the treatment salinity (wt-% NaCl) on the x-axis and counts/counts at 2 wt-% NaCl on the y-axis. Excellent reduction of Vibrio species was shown already at 8 wt-% NaCl and essentially no Vibrio species were found at salinities at or above 12 wt-% NaCl after treatment for 24 hours.

### Example 4

A marine sediment sample was collected from Pond 3 in a traditional shrimp aquaculture farm located in Sonora, Mexico. The sample comprised wet, black mud from the bottom sediments of the shrimp pond that contained organic matter. The 20 liter sample was collected and immediately placed on ice until subsamples of the marine sample were withdrawn the following day. Test tubes were prepared with salinity increments of 0, 2, 8, 12, 18, and 25 wt-% NaCl by using a saturated solution of marine brine and diluting with fresh water. A total of nine milliliters of each of these salinity increments were placed in a test tube and 1.0 grams of sediment was added to each test tube at the different salinity increments. The test tubes were placed in an orbital incubator operating at 150 rpm for 24 hours and at 30 degrees Celsius.

Thereafter, 100 microliter samples were seeded onto Thiosulfate Citrate Bile Sucrose agar plates (TCBS) by extension, and incubated for 24 hours at 30 degrees Celsius. After this incubation period, the Colony Forming Unit (CFUs) per gram, or CFU's/gram, were counted. The Vibrio bacteria incubated on TCBS agar produce either yellow or green colonies, depending if they can ferment sucrose, or not. When sucrose fermentation occurs, yellow colonies are produced by species such as Vibrio cholera. Vibrio species known to be shrimp pathogens such as Vibrio parahaemolyticus, produce green colonies when incubated on TCBS agar. In all of the colonies observed, about 95% of the colonies during analysis were yellow, and about 5% of the colonies were green. The kill rate at the different salinities was based on the CFU/gram count counted at 2 wt-% NaCl. The kill rate was computed as: (1-(CFU/gram at the salinity of interest divided by the CFU/gram at 2 wt-% NaCl))×100%. At 8, 12, and 18 wt-% NaCl, the percentage reduction in CFU's/gram was 60.2, 79.0, and 99.7%, respectively. No CFU's/gram were observed at 25 wt-% NaCl-thus the percentage reduction in CFU's/gram were essentially 100%, within measurement accuracy at these salinities.

A graphical representation of the results is shown in FIG. 3 with crosses, the treatment salinity (wt-% NaCl) on the x-axis and counts/counts at 2 wt-% NaCl on the y-axis. Excellent reduction of Vibrio species was shown already at 8 wt-% NaCl and essentially no Vibrio species were found at salinities at or above 18 wt-% NaCl after treatment for 24 hours.

### REFERENCES CITED

### U.S. Patent Documents

U.S. Pat. No. 5,692,455 Fluidized bed production of oysters and other filter feeding bivalve mollusks using shrimp pond water Wang; Jaw Kai (Honolulu, HI)
U.S. Pat. No. 5,732,654 Open air mariculture system and method of culturing marine animals, Perez; Carlos E. (Guayaquil, EC), Hunter; Max L. (Guayaquil, EC)
U.S. Pat. No. 6,440,466 Composition for treating white spot syndrome virus (WSSV) infected tiger shrimp Penaeus monodon and a process for preparation thereof, Desai; Ulhas Manohar (Goa, IN), Achuthankutty; Chittur Thelakkat (Goa, IN), Sreepada; Rayadurga Anantha (Goa, IN)
U.S. Pat. No. 6,518,317 Antiviral Agents, Sagawa, H. et al. (Otsu, JP)
U.S. Pat. No. 6,615,767 Aquaculture method and system for producing aquatic species Untermeyer; Thomas C. (Lakehills, TX), Williams; Bill G. (Waco, TX), Easterling; Gerald (Carrollton, TX)
U.S. Pat. No. 6,986,323 Inland Aquaculture of Marine Life Using Water From a Saline Aquaifer, Ayers, Andrew D., (Payson, AZ)
U.S. Pat. No. 7,140,323 Reducing the level of bacteria and viruses in aquaculture, Ashworth; David Wilson (Lowdham, GB), Benz; Angelika (Romerberg, DE), Zeller; Dieter (Speyer, DE)
U.S. Pat. No. 7,306,818 Method for preventing and/or treating viral infection in aquatic animals, Su; Wei-Chih (Tainan County, TW), Hsiao; Hsiung (Tainan County, TW), Yang; Chiung-Hua (Tainan County, TW)
U.S. Pat. No. 7,652,050 Methods for increasing the survival of aquatic animals infected with an aquatic virus, Sandino; Ana Maria (Santiago, CL), Mlynarz; Geraldine Z. (Santiago, CL)

### OTHER PUBLICATIONS

Walker Peter J., Winton James R., “Emerging viral diseases of fish and shrimp” 1 CSIRO Livestock Industries, Australian Animal Health Laboratory (AAHL), 5 Portarlington Road, Geelong, Victoria, Australia 2 USGS Western Fisheries Research Center, 6505 NE 65^{th} Street, Seattle, Washington, USA (2010)
Blake,P A., M, H. Merson, R. E. Weaver, D. G. Hollis, and P. C. Heublein. (1979) Disease Caused by a Marine Vibrio—Clinical Characteristics and Epidemiology. N Engl J Med, 300:1-5. DOI: 10.1056/NEJM197901043000101
Walker, P. J., and J. Winton, “Emerging viral diseases of fish and shrimp” Vet. Res. (2010) 41:51
Boyd, Claude E. and Musig, Yont, “Shrimp Pond Effluents: Observations of the Nature of the Problem on Commercial Farms”, 1992 Proceedings of the Special Session on Shrimp Farming, World Aquaculture Society, Baton Rouge, LA Wyban, J., Editor
Wijffels, Rene H. and Barbosa, Maria J., “An Outlook on Microalgal Biofuels”, Science, Vol 329, Aug. 13, 2010 pp 796-799
Walker Peter J., Winton James R., “Diseases of Fish and Shrimp”, Vet. Res. (2010) 41:51
Gill, Tom A., “Waste from Processing Aquatic Animals and Animal Products: Implications on aquatic pathogen transfer”, Series Title: Fisheries Circular C956, 2000, 26 pg, X9199/E (2000)
Johannes, R. E. and Satomi, Masako, “Composition and Nutritive Value of Fecal Pellets of a Marine Crustacean”, Limnol. Oceanogr. 11:191-197 (1996)
Vaiphasa, C., de Boer, W. F., Skidmore, A. K., Panitchart, S., Vaiphasa, T., Bamrongrugsa, N., Santitamnont, P., “Impact of solid shrimp pond waste materials on mangrove growth and mortality: a case study from Pak Phanang, Thailand”, Hydrobiologia (2007) 591:47-57
Sandifer, P. A. and Hopkins, J. S., “Conceptual Design of a Sustainable Pond-based Shrimp Culture System,” Aquacultural Engineering, Vol. 15, No 1, pp41-52, Elsevier Science Limited, Great Britain, 1996.
Houston, J. E. and Nieto, Amelia “Regional Shrimp Market Responses to Domestic Landings and Imports,” Journal of Food Distribution Research, pp, 99-107, February 1988.
Lightner DV (2003) The penaeid shrimp viral pandemics due to IHHNV, WSSV, TSV and YHV: history in the Americas and current status. In: Proceedings of the 32nd Joint UJNR Aquaculture Panel Symposium, Davis and Santa Barbara, California, USA, pp. 17-20.
Fegan, Second FAO Symposium on AHPND Bangkok, 2016
Loc Tran. Second FAO Symposium on AHPND Bangkok 2016

1. A process for growing both aquatic animals and algae within the same aquaculture pond and/or reducing infestation species in pond bottom solids of the aquaculture pond, wherein the process comprises:
growing aquatic animals in the aquaculture pond in a first growth medium;
harvesting the aquatic animals from the aquaculture pond;
after the harvesting of the aquatic animals, providing a second growth medium in the aquaculture pond, wherein the second growth medium comprises a greater salinity than the salinity of the first growth medium, and the salinity of the second growth medium is at least 7 wt-%; and
growing algae in the aquaculture pond in the second growth medium.

2. The process of claim 1, further comprising re-introducing an amount of additional first growth medium in the aquaculture pond and re-introducing aquatic animals into the aquaculture pond for further growth of aquatic animals.

3. The process of claim 1, further comprising recycling at least a portion of the first growth medium for use in the second growth medium.

4. The process of claim 1, wherein the process further comprises:
draining the aquaculture pond after harvesting the aquatic animals and before the growing of algae in the aquaculture pond; and
following the draining, filling the drained aquaculture pond with the second growth medium for the growing of the algae.

5. The process of claim 4, wherein the pond bottom solids of the drained aquaculture pond are allowed to dry, become oxidized, and/or be exposed to UV and/or atmospheric air prior to the filling the drained aquaculture pond with the second growth medium to reduce infestation species in the pond bottom solids.

6. The process of claim 1, wherein the algae are grown in the aquaculture pond for at least about 24 hours.

7. The process of claim 1, further comprising increasing a salinity of the first growth medium to provide at least a portion of the second growth medium.

8. The process of claim 1, wherein the method further comprises:
after the growing of the algae, directing the second growth medium from the aquaculture pond to one or more additional aquaculture ponds, such as one or more drained additional aquaculture ponds, to produce one or more saline-treated additional aquaculture ponds,
reducing a salinity of the second growth medium to generate first growth medium in the one or more saline-treated additional aquaculture ponds; and
growing aquatic animals in the first growth medium of the one or more saline-treated additional aquaculture ponds.

9. The process of claim 1, wherein the method further comprises:
removing at least some of the algae and/or the second growth medium from the aquaculture pond after growing algae in the aquaculture pond;
after the removing, providing the aquaculture pond with the additional amount of the first growth medium; and
re-introducing aquatic animals into the aquaculture pond.

10. The process of claim 1, further comprising, after the growing of algae in the aquaculture pond, discharging at least a portion of the second growth medium to an open body of water as a saline-treated stream.

11. The process of claim 1, wherein the harvesting of aquatic animals is done by filtering the first growth medium to obtain a retentate comprising the aquatic animals and a filtrate comprising the remaining first growth medium, and wherein the process further comprises:
removing the retentate comprising the aquatic animals from the aquaculture pond;
increasing a salinity of the filtrate comprising the remaining first growth medium to generate the second growth medium; and
directing the second growth medium to the aquaculture pond for the growing of the algae in the aquaculture pond.

12. A system for consecutively growing aquatic animals and algae in the same aquaculture pond comprising:
an aquaculture pond;
an aquatic animal harvester in fluid communication with the aquaculture pond for growing aquatic animals; and
an algal harvester in fluid communication with the aquaculture pond for growing algae.

13. The system of claim 12, wherein the aquaculture pond comprises a first growth medium therein for growing the aquatic animals in a first stage and a second growth medium for growing algae in the aquaculture pond in a second stage following the first stage, wherein the second growth medium comprises a greater salinity than the first growth medium and optionally the salinity of the second growth medium is at least about 7 wt-%.

14. The system of claim 12, further comprising a flow controller disposed within the aquaculture pond, the flow controller being configured to selectively harvest the aquatic animals from the aquaculture pond.

15. The system of claim 12, wherein the system further comprises one or more additional aquaculture ponds in fluid communication with the aquaculture pond.

16. The system of claim 12, further comprising a recycle conduit extending directly or indirectly from an outlet of the aquaculture pond to an inlet of the aquaculture pond to recycle at least a portion of the first growth medium or the second growth medium for use in the aquaculture pond.

17. The system of claim 12, wherein the system further comprises a source of salinity arranged for providing the second growth medium with a predetermined salinity.

18. The system of claim 12, wherein the system further comprises a source of algal nutrients arranged for providing the second growth medium with additional algal nutrients for the growth of the algae.

19. The system of claim 12, wherein the system further comprises a source of additional aqueous medium arranged for providing the first growth medium with a predetermined salinity, wherein the additional aqueous medium comprises a member selected from the group consisting of fresh water, seawater, brackish water, and a brine medium having a salinity greater than seawater.

20. The system of claim 12, further comprising a recycle conduit arranged for delivering an aqueous medium from an outlet of the one or more algal or aquatic animal harvesters to an inlet of the aquaculture pond to provide at least a portion of the first growth medium or the second growth medium to the aquaculture pond.

21. The system of claim 12, wherein the algae comprises:
one or more microalgal species selected from the group consisting of Amphora sp., Anabaena sp., Anabaena flos-aquae, Ankistrodesmus falcatus, Arthrospira sp., Arthrospira (Spirulina) obliquus, Arthrospira (Spirulina) platensis, Botryococcus braunii, Ceramium sp., Chaetoceros gracilis, Chlamydomonas sp., Chlamydomonas mexicana, Chlamydomonas reinhardtii, Chlorella sp., Chlorella fusca, Chlorella protothecoides, Chlorella pyrenoidosa, Chlorella stigmataphora, Chlorella vulgaris, Chlorella zofingiensis, Chlorococcum citriforme, Chlorococcum littorale, Closterium sp., Coccolithus huxleyi, Cosmarium sp., Crypthecoddinium cohnii, Cryptomonas sp., Cyclotella cryptica, Cyclotella nana, Dunaliella sp., Dunaliella bardawil, Dunaliella salina, Dunaliella tertiolecta, Dunaliella viridis, Euglena gracilis, Fragilaria, Fragilaria sublinearis, Gracilaria, Haematococcus pluvialis, Hantzschia, Isochrysis galbana, Microcystis sp., Monochrysis lutheri, Muriellopsis sp., Nannochloris sp., Nannochloropsis sp., Nannochloropsis salina, Navicula sp., Navicula saprophila, Neochloris oleoabundans, Neospongiococcum gelatinosum, Nitzschia laevis, Nitzschia alba, Nitzschia communis, Nitzschia paleacea, Nitzschia closterium, Nitzschia palea, Nostoc commune, Nostoc flagellaforme, Pavlova gyrens, Peridinium, Phaeodactylum tricornutum, Pleurochrysis carterae, Porphyra sp., Porphyridium aerugineum, Porphyridium cruentum, Prymnesium, Prymnesium paruum, Pseudochoricystis ellipsoidea, Rhodomonas sp., Scenedesmus sp., Scenedesmus braziliensis, Scenedesmus obliquus, Scenedesmus quadricauda, Scenedesmus acutus, Scenedesmus dimorphus, Schizochytrium sp., Scytonema, Skeletonema costatum, Spirogyra, Schiochytrium limacinum, Stichococcus bacillaris, Synechoccus, Tetraselmis sp., Tolypothrix sp., genetically-engineered varieties thereof, and any combinations thereof; or
one or more prokaryotes selected from the group consisting of Aphanothece halophytica, Microcoleus chthonoplastes, M. lyngbyaceus, Spirulina major, S. platensis, Nodularia spumigena, Dactylococcopsis salina, Synechocystis DUN52, PCC 6803, Synechococcus PCC 7418, Phormidium spp., Oscillatoria spp., Lyngbya spp., Halospirulina tapeticola, Microcystis spp., Nostoc spp., and Aphanocapsa spp.; or
one or more eukaryotes selected from the group consisting of Dunaliella spp., Dangeardinella saltitrix, Chlorella vulgaris, Navicula spp., Amphora spp., and Amphora spp.; or
genetically-engineered varieties of any of the above; or
any combinations thereof.

22. The system of claim 12, wherein the salinity of the second growth medium is at least about 8 wt-%, at least about 9 wt-%, at least about 10 wt-%, at least about 11 wt-%, at least about 12 wt-%, at least about 13 wt-%, at least about 14 wt-%, at least about 15 wt-%, at least about 16 wt-%, at least about 17 wt-%, at least about 18 wt-%, at least about 19 wt-%, at least about 20 wt-%, at least about 21 wt-%, at least about 22 wt-%, at least about 23 wt-%, at least about 24 wt-%, or at least about 25 wt-%.

23. The system of claim 12, wherein the first medium comprises a salinity of from 0 to about 5 wt-%.

24. The system of claim 12, wherein the aquaculture pond is an open aquaculture pond.

25. The system of claim 12, wherein the aquatic animals are selected from the group consisting of crustaceans, shrimps, fishes, molluscs, shellfishes, and any combination thereof.

26. The system of claim 12, wherein the size of the aquaculture pond is about 0.1—about 1000 hectares, about 0.1—about 200 hectares, about 0.1—about 100 hectares, about 0.1—about 20 hectares, about 1—about 50 hectares, about 1—about 20 hectares, about 1—about 10 hectares, about 1—about 5 hectares, or less than about 1 hectare or about 0.1 hectare.

27. The system of claim 12, wherein the system is configured to carry out the process.

28. Use of the system of claim 12 for growing aquatic animals and algae consecutively in the same aquaculture pond, wherein the aquatic animals are harvested before growing algae.
